(19) United States
(12) Patent Application Publication
(10) Pub. No.: US 2004/0057958 A1
(43) Pub. Date: Mar. 25, 2004

(54) Immunogenicity-enhancing carriers and compositions thereof and methods of using the same

(76) Inventors: David W. Waggoner JR., Seattle, WA (US); Michael E. Coon, Seattle, WA (US)

Correspondence Address: DONALD W. WYATT, CELL THERAPEUTICS, INC., 501 ELLIOTT AVENUE WEST, #400, SEATTLE, WA, 98119, US

(21) Appl. No.: 10/441,944

(22) Filed: May 19, 2003

Related U.S. Application Data
(60) Provisional application No. 60/381,550, filed on May 17, 2002.

(51) Int. Cl. A61K 39/00; C07K 14/00

(52) U.S. Cl. 424/184.1; 530/403; 514/54

(57) ABSTRACT

The present invention is directed to compositions comprising a substantially non-antigenic carrier associated with an antigen and the use of such compositions to enhance the immunogenicity of the associated antigen. In addition, the compositions of the invention may be used to generate an immune response directed predominantly to an antigen associated with a carrier. Specific carriers of the invention include homopolymers and copolymers of polyamino acids. Compositions of the invention are used according to the invention to elicit or enhance an immune response directed against an antigen and may be used for the prevention and treatment of infection and disease, for example. Additionally, compositions of the invention are useful for generating an antibodies specific for an antigen and, accordingly, may be used to generate antigen-specific antibodies suitable for the diagnosis or treatment of infection and disease.

### BACKGROUND OF THE INVENTION

1. Field of the Invention

This invention generally relates to compositions and methods for enhancing the immunogenicity of an antigen or hapten. More particularly, this invention is directed to compositions comprising a homopolymer or copolymer of polyamino acids as carriers and/or adjuvants and methods of using said composition to induce an immune response.

2. Description of the Related Art

It is well known that when animals are immunized with small organic compounds (haptens) conjugated to large proteins (carriers), the conjugate induces a humoral immune response with antibodies formed both to hapten epitopes and to unaltered epitopes on the carrier protein. See Kuby, Immunology (2^{nd} Edition, FREEMAN), p330, 1991. Many biologically important substances, including drugs, peptide hormones, and steroid hormones, can function as haptens. Common carrier proteins include bovine gamma-globulin (BGG), bovine serum albumin (BSA), keyhole limpet hemocyanin (KLH), ovalbumin (OVA), and human gamma-globulin (HGG). Id. However, the common carrier proteins listed above are themselves antigenic, and, therefore, cannot be used to induce an immune response solely to conjugated haptens.

Furthermore, many protein, and most peptide, carbohydrate, and lipid antigens, administered alone, do not elicit a sufficient antibody response to confer immunity. The immune response to weakly immunogenic antigens can be significantly enhanced if the antigens are co-administered with adjuvants. An adjuvant is any substance that enhances the immunogenicity of substances mixed with it. Adjuvants enhance the immunogenicity of an antigen but are not necessarily immunogenic themselves.

The best known adjuvants include Freund's complete adjuvant (FCA), an emulsion containing mineral oil and killed mycobacteria in saline, Freund's incomplete adjuvant (FIA), omitting the mycobacteria, N-acetylmuramyl-L-alanyl-D-isoglutamine (commonly known as muramyl dipeptide or “MDP”) and lipopolysaccharide (LPS). However, their clinical use is limited by their toxicity. Currently, the only FDA-approved adjuvant for use in humans is aluminum salts (Alum) which are used to “depot” antigens by precipitation of the antigens.

Every day additional epitopes are identified from diseased tissues and disease causing agents including microbes, such as viruses, bacteria and parasites, tumor cells, and autoimmune diseases. Thus, there is a need in the art to develop ways by which a specific immune response to an antigenic epitope is induced.

The present invention satisfies this need and provides other related advantages as set forth in more detail below.

### BRIEF SUMMARY OF THE INVENTION

The present invention provides compositions and methods useful for generating or enhancing an immune response directed against or specific to an antigen. In certain embodiments of the invention, the compositions and methods of the invention are used to stimulate or enhance an immune response directed against a substantially non-immunogenic antigen or hapten, although the invention is also useful for enhancing an immune response to an immunogenic antigen or hapten. In one embodiment of the invention, the composition comprises an immunogenic carrier that enhances the immunogenicity of a conjugated antigen or hapten. In certain embodiments, the carrier is substantially non-antigenic, although the carrier may also be antigenic.

In one aspect, the invention provides a composition for enhancing the immunogenicity of a hapten or an antigenic compound comprising a substantially non-antigenic carrier and the antigen or hapten, wherein the carrier is associated with the antigen or hapten.

The hapten or antigenic compound may be conjugated or associated with the carrier by any means known available or known in the art. In one embodiment, the association of the hapten or antigenic compound and the carrier is by charge-charge interaction. In another embodiment, the association of the hapten or antigenic compound and the carrier is a covalent bond. The covalent bond may be a peptide bond.

In one embodiment of the invention, the carrier is a polymer. In certain embodiments, the polymer is a polyamino acid polymer, a polyanionic amino acid polymer, or a polyglutamate polymer. In one embodiment, the polymer is a polyanionic amino acid, polyglycolide, polylactide, poly(p-dioxanone), polycaprolactone, polyhydroxyalkanoate, poly(propylene fumarate), poly(ortho ester), polyanhydride, polyphosphazene, poly(alkylcyanoacrylate), poloxamer, polyglutamate, or polyethylene glycol.

In a related embodiment, the polymer is biodegradable. In specific embodiments, the polymer has a molecular weight of between ten and 5,000 KD, between 100 and 1,000 KD, or about 600 KD.

In another embodiment of the invention, the composition further comprises a physiologically acceptable excipient, diluent, or adjuvant. In specific embodiments, an adjuvant is a liposome, oily phase, Freund's adjuvant, inorganic salt, cytokine, chemokine, growth factor, angiogenic factor, apoptosis inhibitor, hormone, immunomodulator, plasmid DNA, polyinosine:cytosine, immunostimulatory oligonucleotide, bacterial agent, listeriolysin, streptolysin, mineral oil, non-mineral oil, self-emulsifiable oil, pertussis toxin mutant, saponin, lipopolysaccharide, monophosphoryl lipid A, or N-acetylmuramyl-L-alanyl-D-isoglutamine, or a related compound.

In yet another embodiment of the invention, the hapten or antigenic compound is (1) a live, heat-killed, or chemically attenuated microbes; (2) a fragment, extract, subunit, metabolite, or recombinant construct of a microbe or mammalian protein, glycoprotein or other antigen or epitope; (3) a tumor antigen; or (4) a nucleic acid molecule.

In specific embodiments, the tumor antigen is carcinoembryonic antigen, carcinoembryonic antigen peptide-1 (CAP-1), α-fetoprotein, alkaline phosphatase isoenzyme, prostate-specific antigen, beta subunit of choriogonadotropic hormone, calcitonin, Bence-Jones proteins, aspartyl β-hydroxylase, NY-ESO-1, 707 alanine proline, adenocarcinoma antigen (ART-4), B antigen (BAGE), β-catenin, m-catenin, Bcr-abl, CTL-recognized antigen on melanoma (CAMEL), caspase-8, CDC27, CDK4, cancer/testis antigen, cyclophilin B, differentiation antigens melanoma (DAM-6 and DAM-10), elongation factor 2, Ets, glycoprotein 250, G antigen, N-acetylglucosaminyltransferase V, glycoprotein 100 kD, helicose antigen, human epidermal receptor-2/neurological (HER2-neu), HLA-A2 R170I, human papilloma virus E7, heat shock protein 70—2, mutated, human signet ring tumor—2, human telomerase reverse transcriptase, intestinal carboxyl esterase, L antigen, low density lipid receptor/GDP-L-fucose:-D-galactosidase 2-L-fucosyltransferase, melanoma antigen, Melanoma antigen A, melanocortin 1 receptor, myosin (mutated), mucin 1, melanoma ubiquitous mutated 1, 2, 3, NA cDNA clone of patient M88, New York—esophageous 1, protein 15, minor bcr-abl, promyelocytic leukaemia/retinoic acid receptor (Pml/RAR), preferentially expressed antigen of melanoma, prostate-specific antigen, prostate-specific membrane antigen, renal antigen, renal ubiquitous 1 or 2, sarcoma antigen, squamous antigen rejecting tumor 1 or 3, translocation Ets-family leukemia/acute myeloid leukemia 1 (TEL/AML1), triosephosphate isomerase (mutated), gp75, tyrosinase related protein 2, TRP-2/intron, or Wilms' tumor gene.

In one embodiment, the antigenic compound is derived from an agent selected from the group consisting of a virus, a fungus, a bacteria, a diseased tissue, or a hapten. In a specific embodiment, the agent is a virus. In another specific embodiment, the diseased tissue is a tumor. In certain embodiments, the antigenic compound is a peptide, protein, polysaccharide, or hapten.

In a certain embodiment, the invention provides an immunogenic composition for enhancing the immunogenicity of an antigenic compound, comprising a substantially non-antigenic, biodegradable and soluble carrier, and the antigenic compound; wherein the antigenic compound is associated with the carrier. In one embodiment, the antigenic compound is covalently conjugated to the carrier. In one embodiment, the antigenic compound is associated with the carrier by charge interactions.

In certain embodiments, the composition of the invention further comprise a surfactant. In specific embodiment, the surfactant is a polyethylene glycol, including PEG 200, 300, 400, 600 and 900, Span®, Arlacel®, Tween®, including Tween® 80, Myrj®, Brij®, polyoxyethylene, polyol fatty acid ester, polyoxyethylene ether, polyoxypropylene fatty ether, bee's wax derivative containing polyoxyethylene, polyoxyethylene lanolin derivative, polyoxyethylene fatty glyceride, glycerol fatty acid ester, polyoxyethylene acid alcohol, or ether derivatives of long-chain fatty acids of 12-21 carbon atoms.

In a related embodiment, the invention includes an immunogenic composition for enhancing the immunogenicity of an antigenic compound, comprising a substantially non-antigenic polyanionic amino acid polymer, and an antigenic compound; wherein the antigenic compound is a peptide, polypeptide or a hapten, and wherein said peptide, polypeptide or a hapten is conjugated to the polyanionic amino acid polymer. In specific embodiments, the polyanionic amino acid polymer is a homopolymer or a polyglutamate homopolymer. In related embodiments, the homopolymer has a molecular weight of between ten and 5,000 KD, between 100 and 1,000 KD, or about 600 kDa.

In one embodiment, the polymer is conjugated to the antigenic compound by a peptide bond.

In a related embodiment, the invention provides a method for eliciting, inducing, or enhancing an immune response to an antigenic compound, comprising administering to a mammal a composition of the invention.

In one embodiment, the invention provides a method of eliciting, inducing or enhancing an immune response in a mammal, comprising administering to the mammal an effective amount of a composition of the invention.

In certain embodiments, a method of the invention elicits, enhances, or induces a protective immune response.

In specific embodiments, a method of the invention elicits, enhances, or induces a cellular or cell-mediated immune response, while in other embodiments, a method of the invention elicits, enhances, or induces a humoral response. A method of the invention may also elicit, enhance, or induce both a humoral and cellular immune response.

In another embodiment, the invention provides a method for treating or ameliorating a viral infection in a mammal, comprising administering to said mammal an effective amount of a composition of the invention.

In a related embodiment, the invention provides a method for treating or ameliorating a pathology in a mammal, comprising administering to said mammal an effective amount of a composition of the invention.

In another embodiment, the invention provides a mammal inoculated with the composition of the invention.

In a related embodiment, the invention provides a kit comprising a substantially non-antigenic carrier of the invention and instructions for associating or conjugating said carrier with an antigenic compound or hapten.

### DETAILED DESCRIPTION OF THE INVENTION

The present invention may be understood more readily by reference to the following detailed description of certain embodiments of the invention and the Examples included herein.

Throughout this application, where publications, patents, and patent applications are referenced, the disclosures of these publications, patents, and patent applications are hereby incorporated by reference, in their entireties, into this application in order to more fully describe the state of the art to which this invention pertains.

Definitions

Prior to setting forth details of the present invention, it will be useful to an understanding thereof to set forth definitions of several terms that your used herein.

The term “adjuvant”, as used herein, refers to any substance or mixture of substances that increases or diversifies the immune response to an antigenic compound.

“Allergy” or “atopy”, as used herein, refers to an increased tendency to IgE-based sensitivity resulting in production of specific IgE antibody to an immunogen, particularly to common environmental allergens such as insect venom, house dust mite, pollens, molds, or animal danders.

The term “amino acid”, as used herein, refers to both natural and synthetic amino acids, and includes, but is not limited to, alanyl, valinyl, leucinyl, isoleucinyl, prolinyl, phenylalaninyl, tryptophanyl, methioninyl, glycinyl, serinyl, threoninyl, cysteinyl, tyrosinyl, asparaginyl, glutaminyl, aspartoyl, glutaoyl, lysinyl, argininyl, and histidinyl.

The term “animal”, as used herein, includes humans and all domestic and wild mammals and fowl, including, without limitation, cattle, horses, swine, sheep, goats, dogs, cats, rabbits, deer, mink, chickens, ducks, geese, turkeys, game hens, and the like.

The term “antibody”, as used herein, includes polyclonal and monoclonal antibodies, as well as antigenic compound-binding fragments of such antibodies including Fab, F(ab′)₂, Fd, Fv fragments, and single chain derivatives of the same. “Antibody” also includes cell-associated antibodies, such as Ig receptors, for example. In addition, the term “antibody” includes naturally occurring antibodies, as well as non-naturally occurring antibodies, including, for example, chimeric, bifunctional, and humanized antibodies, and related synthetic isoforms.

As used herein, the term “antigenic compound” refers to any substance that can be recognized by an antibody under appropriate or physiological conditions. “Antigenic compound” includes any antigen or hapten.

An “antigen”, as used herein, refers to a substance that binds specifically or selectively to an antibody or a T-cell receptor, under appropriate conditions. Thus, an antigen may be a target of an acquired (i.e., adaptive) immune response, but it may or may not itself induce an acquired immune response.

A “hapten”, as used herein, refers to an antigenic molecule that is substantially non-immunogenic by itself but can become immunogenic when associated with a larger molecule, sometimes referred to as a “carrier.” Preferably, the association is by covalent conjugation, but it can be by charge interactions and other suitable mechanisms. In preferred embodiments, the larger molecule may be substantially non-antigenic.

The term “substantially non-antigenic” refers to a substance that does not substantially bind specifically or selectively to an antibody or a T-cell receptor, under appropriate conditions. For example, a substantially non-antigenic substance is one that does not elicit a statistically significant antigenic response from a vertebrate as detected by ELISA assay as compared to a positive control. In certain embodiments, the ELISA assay is a sandwich ELISA assay or a direct ELISA assay.

The term “biodegradable”, as used herein, refers to a carrier that can be degraded in vivo.

The term “biological activity”, as used herein, refers to a molecule having a biological or physiological effect or response in a vertebrate subject. Adjuvant activity is an example of a biological activity. Activating or inducing production of other biological molecules having adjuvant activity is also a contemplated biological activity.

The terms “cell-mediated immunity” and “cell-mediated immune response” refer to the immunological defense provided by lymphocytes, such as that defense provided by T cell lymphocytes when they come into close proximity to target cells. A cell-mediated immune response also comprises lymphocyte proliferation, recruitment, invasion, and activation. When “lymphocyte proliferation” is measured, the ability of lymphocytes to proliferate in response to specific antigen is measured. Lymphocyte proliferation is meant to refer to B cell, T-helper cell or cytotoxic T-lymphocyte (CTL) cell proliferation.

The term “CTL response” refers to the ability of an antigen-responsive T-cell to lyse and kill a cell expressing the specific antigen. Standard, art-recognized CTL assays are performed to measure CTL activity.

An “effective amount of an antigenic compound” refers to an amount of antigenic compound which, in optional combination with an adjuvant, will cause the subject to produce a specific immunological response to the antigenic compound.

As used herein, an “epitope” refers to the site on an antigen that is recognized and bound by a particular antibody or T-cell receptor. The minimal size of a protein epitope, as defined herein, is about five amino acids, and a protein epitope typically comprises at least eight amino acids. It is to be noted, however, that an epitope might comprise a portion of an antigen other than the amino acid sequence, e.g., a carbohydrate moiety or a lipid moiety. Furthermore, an epitope may be discontinuous, i.e., it comprises amino acid residues that are not adjacent in the polypeptide but are brought together into an epitope by way of the secondary, tertiary, or quaternary structure of the protein.

The terms “humoral immunity” or “humoral immune response” refers to the form of immunity mediated by antibody molecules secreted in response to immunogenic stimulation, as well as B cell recruitment of cellular and innate responses.

The term “immune response” refers to any response to an immunogenic compound by the immune system of a vertebrate subject. Exemplary immune responses include, but not limited to cellular as well as local and systemic humoral immunity, such as CTL responses, including antigen-specific induction of CD8+ CTLs, helper T-cell responses, including T-cell proliferative responses and cytokine release, and B-cell responses including antibody response.

The term “inducing an immune response” refers to administration of an immunogenic compound or a nucleic acid encoding the immunogenic compound, wherein an immune response is effected, i.e., stimulated, initiated or induced.

The term “potentiating an immune response” refers to administration of an immunogenic compound or a nucleic acid encoding the antigenic compound, wherein a preexisting immune response is improved, furthered, supplemented, amplified, increased or prolonged. The immunogenic compound may be, for example, a composition comprising an antigen or hapten associated or conjugated to a carrier, with or without an adjuvant.

The term “immunogenic amount” refers to an amount of a compound sufficient to stimulate an immune response, when administered according to the invention. The amount of a compound necessary to provide an immunogenic amount is readily determined by one of ordinary skill in the art, e.g., by preparing a series of compositions of the invention with varying concentrations of antigenic compound, administering such compositions to suitable laboratory animals (e.g., guinea pigs), and assaying the resulting immune response by measuring serum antibody titer, antigen-induced swelling in the skin, and the like.

The term “immunopotentiating amount” refers to the amount of the carrier needed to effect an increase in antibody titer and/or cell mediated immunity when administered with an antigenic compound in a composition of the invention, as compared with the titer level observed in the absence of the carrier. The immunopotentiating amount may easily be determined by one of ordinary skill in the art.

As used herein, the term “mixing” includes any method to combine the components of the composition; such methods include, but are not limited to, blending, dispersing, dissolving, emulsifying, coagulating, suspending, or otherwise physically combining the components of the composition.

The term “optionally”, as used herein, refers to an instance wherein the subsequently described event or circumstances may or may not occur, and that the description includes instances where said event or circumstances occurs and instances in which it does not occur.

The term “pharmaceutically acceptable salt” refers to an acid addition salt of a subject compound which possesses the desired pharmacological activity and which is neither biologically nor otherwise undesirable. This salt is formed with an inorganic acid such as hydrochloric acid, hydrobromic acid, sulfuric acid, nitric acid, or phosphoric acid; or an organic acid such as acetic acid, propionic acid, glycolic acid, pyruvic acid, malonic acid, succinic acid, malic acid, maleic acid, fumaric acid, tartaric acid, citric acid, benzoic acid, cinnamic acid, mandelic acid, methanesulfonic acid, ethanesulfonic acid, p-toluenesulfonic acid, and the like.

The term “poly (amino acid) polymer”, as used herein, refers to a polymer comprised of naturally occurring or synthetic amino acids either as a heteropolymer or homopolymer. The amino acids need not be polymerized through peptide bonds but may be bound in any fashion that allows amino acid monomers to be bound sequentially.

The term “poly (anionic amino acid) polymer”, as used herein, refers to a polymer comprised of amino acid monomers such that the polymer exhibits a net anionic character.

The terms “poly-glutamic acid” or “poly-glutamic acids” include poly (1-glutamic acid), poly (d-glutamic acid) and poly (dl-glutamic acid), the terms “a poly-aspartic acid” or “poly-aspartic acids” include poly (1-aspartic acid), poly (d-aspartic acid), and poly (dl-aspartic acid), the terms “a poly-lysine” or “poly-lysines” include poly (1-lysine), poly (d-lysine), and poly (dl-lysine), the terms “a poly-serine” or “poly-serines” include poly (1-serine), poly (d-serine), and poly (dl-serine), the terms “a poly-glycine” or “poly-glycines” include poly (1-glycine), poly (d-glycine), and poly (dl-glycine), the terms “a poly-alanine” or “poly-alanines” include poly (l-alanine), poly (d-alanine), and poly (dl-alanine), and the terms “a poly-cysteine” or “poly-cysteines” include poly (1-cysteine), poly (d-cysteine), and poly (dl-cysteine). The terms “a water soluble polyamino acid”, “water soluble polyamino acids”, or “water soluble polymer of amino acids” include, but are not limited to, poly-glutamic acid, poly-aspartic acid, poly-lysine, and amino acid chains comprising mixtures of glutamic acid, aspartic acid, and/or lysine.

In certain embodiments, the terms “a water soluble polyamino acid”, “water soluble polyamino acids”, or “water soluble polymer of amino acids” include amino acid chains comprising combinations of glutamic acid and/or aspartic acid and/or lysine, of either d and/or I isomer conformation. In certain prefered embodiments, such a “water soluble polyamino acid” contains one or more glutamic acid, aspartic acid, and/or lysine residues.

The term “systemic immune response” is meant to refer to an immune response in the lymph node-, spleen-, or gut-associated lymphoid tissues wherein cells, such as B lymphocytes, of the immune system are developed. For example, a systemic immune response can comprise the production of serum IgG's. Further, systemic immune response refers to antigen-specific antibodies circulating in the blood stream and antigen-specific cells in lymphoid tissue in systemic compartments such as the spleen and lymph nodes. In contrast, the gut-associated lymphoid tissue (GALT) is a component of the mucosal immune system since antigen-specific cells that respond to gut antigens/pathogens are induced and detectable in the GALT.

The term “treatment” as used herein covers any treatment of a disease in vertebrate animal, particularly a human, and includes: (i) preventing the disease from occurring in a subject which may be predisposed to the disease but has not yet been diagnosed as having it; (ii) inhibiting the disease, i.e., arresting its development; or (iii) relieving the disease, i.e., causing regression of the disease. (It should be noted that vaccination may effect regression of a disease where the disease persists due to ineffective antigen recognition by the subject's immune system, where the vaccine effectively presents antigen.)

Carriers

The present invention provides a carrier that enhances the immunogenicity of an antigen, a hapten, or any other antigenic compound that is immunogenic, non-immunogenic, or weakly immunogenic when not associated with the carrier. A desired carrier of the present invention has physiochemical qualities including being non-immunogenic, non-allergenic, or non-antigenic, being metabolizable, being large molecular weight, being soluble, particularly in aqueous physiological solutions, such as phosphate buffered saline, for example, and capable of being conjugated (e.g., covalently bound) or associated (e.g., admixed with or associated through charge-charge interactions) with the antigenic compound.

The invention contemplates the use of a single carrier and the use of mixtures of different carriers. Different carriers include, for example, polymers of different lengths, such, as, for example, two or more different length homopolymers, as well as mixtures of two or more different carriers or polymers of the invention. In one embodiment, it is advantageous to use a single carrier, while in other embodiments, it is advantageous to use a mixture of different carriers. For example, using a single carrier will entail the design and production of only one carrier-hapten complex or fusion, whereas using multiple carriers fused to a single hapten will entail designing and producing multiple carrier-hapten complexes or fusion. However, using more than one carrier may be advantageous if the immune response generated against a particular hapten or epitope varies, such as in magnitude or specificity, for example, depending upon the particular carrier used, and the most optimal carrier is not known or has not yet been experimentally determined.

In certain aspects, the carrier is a polymer, which may be synthetic or natural. Further, the polymer carrier may be substantially non-antigenic or biodegradable, or both. In certain embodiments, the compositions of the present invention may comprise a wide variety of polymers. In one embodiment, the polymers can be a poly(diene), a poly(alkene), a poly(acrylic), a poly(methacrylic), a poly(vinyl ether), a poly(vinyl alcohol), a poly(vinyl ketone), a poly(vinyl halide), a poly(vinyl nitrile), a poly(vinyl ester), a poly(styrene), a poly(carbonate), a poly(ester), a poly(orthoester), a poly(esteramide), a poly(anhydride), a poly(urethane), a poly(amide), a cellulose ether, a cellulose ester, a poly(saccharide), poly(lactide-co-glycolide), a poly(lactide), a poly(glycolide), a copolyoxalate, a polycaprolactone, a poly(lactide-co-caprolactone), a poly(esteramide), a polyorthoester, a poly(a-hydroxybutyric acid), a polyanhydride or a mixture thereof. In particular embodiments, the polymers comprise a poly(lactide-co-glycolide), a poly(lactide), a poly(glycolide), such as polyethylene glycol (PEG), a copolyoxalate, a polycaprolactone, a poly(lactide-co-caprolactone), a poly(esteramide), a polyorthoester, a poly(a-hydroxybutyric acid), a polyanhydride, or a mixture thereof.

The polymers may also be polymers derived from the polymerization of at least one monomer. Thus, in another embodiment, the polymers may be a polymer or oligomer derived from the polymerization or oligomerization of at least one monomer. Examples of suitable monomers include an alpha hydroxycarboxylic acid, a lactone, a diene, an alkene, an acrylate, a methacrylate, a vinyl ether, a vinyl alcohol, a vinyl ketone, a vinyl halide, a vinyl nitrile, a vinyl ester, styrene, a carbonate, an ester, an orthoester, an esteramide, an anhydride, a urethane, an amide, a cellulose ether, a cellulose ester, a saccharide, an alpha hydroxycarboxylic acid, a lactone, an esteramide, or a mixture thereof.

In other embodiments, the polymers are the polymerization products of an alpha hydroxycarboxylic acid, a lactone or a mixture thereof. In yet further embodiments, the alpha hydroxycarboxylic acid comprises glycolic acid, lactic acid, a-hydroxy butyric acid, a-hydroxyisobutyric acid, a-hydroxyvaleric acid, a-hydroxyisovaleric acid, a-hydroxy caproic acid, a-hydroxy-a-ethylbutyric acid, a-hydroxyisocaproic acid, a-hydroxy-3-methylvaleric acid, a-hydroxyheptanoic acid, a-hydroxyoctanoic acid, a-hydroxydecanoic acid, a-hydroxymysristic acid, a-hydroxystearic acid, a-hydroxyligoceric acid or a mixture thereof. In one embodiment, the lactone comprises 3-propiolactone, tetramethyleneglycolide, b-butyrolactone, 4-butyrolactone, pivalactone or mixtures thereof.

In certain embodiments, the carrier is a polymer derived from one or more amino acids. In other embodiments, the polymers are homopolymers or heteropolymers. In certain embodiments, polymers are amino acids or anionic monomers, such as anionic amino acids, for example. One example of an anionic amino acid for the formation of such polymer carriers is glutamic acid. For example, polyglutamate derived from L-glumatic acid, D-glumatic acid or mixtures, e.g., racemates, of these L and D isomers are used. L and/or D glutanyl, aspartly, glycyl, seryl, threonyl, and cysteinyl are all examples of amino acids that may be used according to the invention.

In other embodiments, the polymers are copolymers, such as block, graft or random copolymers, containing glutamic acid. Thus, copolymers of glutamic acid with at least one other (preferably biodegradable) monomer, oligomer or polymer are included. These include, for example, copolymers containing at least one other amino acid, such as aspartic acid, serine, tyrosine, glycine, ethylene glycol, ethylene oxide, (or an oligomer or polymer of any of these) or polyvinyl alcohol. Glutamic acid may, of course, carry one or more substituents and the polymers include those in which a proportion or all of the glutamic acid monomers are substituted. Substituents include, for example, alkyl, hydroxy alkyl, aryl and arylalkyl, commonly with up to 18 carbon atoms per group, or polyethylene glycol attached by ester linkages. The expression “poly (glutamic acid)” and cognate expressions herein are to be construed as covering any of the aforesaid possibilities unless the context otherwise demands.

In certain embodiments, the polymers are poly(amino acids) including, but not limited to poly(l-glutamic acid), poly(d-glutamic acid), poly(dl-glutamic acid), poly(l-aspartic acid), poly(d-aspartic acid), poly(dl-aspartic acid), poly(l-serine), poly(d-serine), poly(dl-serine), poly(l-tyrosine), poly(d-tyrosine), poly(dl-tyrosine), poly(l-glysine), poly(d-glysine), poly(dl-glysine), poly(l-threonine), poly(d-threonine), poly(dl-threonine), poly(d-cysteine), poly(l-cysteine), and poly(dl-cysteine). In further embodiments, the polymers are copolymers, such as block, graft or random copolymers, of the above listed poly(amino acids) with polyethylene glycol, polycaprolactone, polyglycolic acid and polylactic acid, as well as poly(2-hydroxyethyl 1-glutamine), chitosan, carboxymethyl dextran, hyaluronic acid, human serum albumin and alginic acid, with poly-glutamic acids being particularly preferred.

Polymer carriers of the present invention will generally range from about 1,000 kilodaltons molecular weight to less than 10,000,000 kilodaltons. Although usually not more than about 5,000,000 kilodaltons, polymer carriers of invention have no upper limit to their molecular weight. The polymers of the present invention, in certain embodiments, have a molecular weight of about 10 kilodaltons to about 5,000 kilodaltons, including all integer values within this range, including, for example, 100, 200, 300, 500, 1,000, 1,500, 2,000, 2,500, 3,000, 3,500, 4,000, and 4,500 kilodaltons, with certain embodiments comprising polymer carriers having a molecular weight of about 600 kilodaltons.

In additional embodiments, various substitutions of naturally occurring, unusual, or chemically modified amino acids may comprise the amino acid composition of the poly(amino acid) polymer, and particularly the poly(anionic amino acid) polymers and in certain embodiments the poly-glutamic acid polymers, to produce a poly(amino acid) polymer including, but not limited to polyanionic amino acid polymers having like or otherwise desirable characteristics of a carrier of the present invention. Further, homopolymers of the present invention may comprise polymers that are homo-anionic, for example, comprising strictly anionic amino acids without necessarily being structurally identical.

A poly(amino acid) or poly(anionic amino acid) polymer, such as poly-glutamic acid, poly-aspartic acid, poly-serine, poly-tyrosine, poly-glycine, or water soluble amino acid chain or polymer comprising a mixture of glutamic acid, aspartic acid, serine, tyrosine and/or glycine, may, at the lower end of the amino acid substitution range, have 1, 2, 3, 4, 5, 6, 7, 8, 9, 10, 11, 12, 13, 14, 15, 16, 17, 18, 19, 20, 21, 22, 23, 24, 25 or more glutamic acid, aspartic acid, serine, tyrosine or glycine, residues, respectively, substituted by any of the naturally occurring, modified, or unusual amino acids described herein. In other aspects of the invention, a poly(amino acid) homopolymer such as poly-glutamic acid, poly-aspartic acid, poly-serine, poly-tyrosine, poly-glycine, or a poly(amino acid) copolymer comprising a mixture of some or all of these five amino acids may, at the lower end, have about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, to about 25% or more glutamic acid, aspartic acid, serine, tyrosine or glycine residues, respectively, substituted by any of the naturally occurring, modified, or unusual amino acids described herein.

In further aspects of the invention, a poly(amino acid) homopolymer such as poly-glutamic acid, poly-aspartic acid, poly-serine, poly-tyrosine, or poly-glycine may, at the high end of the amino acid substitution range, has about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, to about 50% or so of the glutamic acid, aspartic acid, serine, tyrosine, or glycine residues, respectively, substituted by any of the naturally occurring, modified, or unusual amino acids described herein, as long as the majority of residues comprise glutamic acid and/or aspartic acid and/or serine and/or tyrosine and/or glycine.

In certain aspects, naturally occurring amino acids for use in the present invention as amino acids or substitutions of a poly(amino acids) are alanine, arginine, asparagine, aspartic acid, citrulline, cysteine, glutamine, glycine, histidine, isoleucine, leucine, lysine, methionine, ornithine, phenylalanine, proline, serine, threonine, tryptophan, tyrosine, valine, hydroxy proline, ε-carboxyglutamate, phenylglycine, or O-phosphoserine.

In other aspects, non-naturally occurring amino acids for use in the present invention are β-alanine, α-amino butyric acid, γ-amino butyric acid, γ-(aminophenyl) butyric acid, α-amino isobutyric acid, citrulline, ε-amino caproic acid, 7-amino heptanoic acid, β-aspartic acid, aminobenzoic acid, aminophenyl acetic acid, aminophenyl butyric acid, γ-glutamic acid, ε-lysine, methionine sulfone, norleucine, norvaline, ornithine, d-ornithine, p-nitro-phenylalanine, hydroxy proline, 1,2,3,4,-tetrahydroisoquinoline-3-carboxylic acid, and thioproline.

Amino acid substitutions are generally based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all a similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as biologically functional equivalents.

To effect more quantitative changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein, and correspondingly a poly(amino acid), is generally understood in the art (Kyte & Doolittle, 1982, incorporated herein by reference). It is known that certain amino acids may be substituted for other amino acids having a similar hydropathic index or score and still retain a similar biological activity. In making changes based upon the hydropathic index, the substitution of amino acids whose hydropathic indices are within +/−2 is preferred, those which are within +/−1 are particularly preferred, and those within +/−0.5 are even more particularly preferred.

It is also understood in the art that the substitution of like amino acids can be made effectively on the basis of hydrophilicity. As detailed in U.S. Pat. No. 4,554,101, the following hydrophilicity values have been assigned to amino acid residues: arginine (+3.0); lysine (+3.0); aspartate (+3.0+/−1); glutamate (+3.0+/−1); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, the substitution of amino acids whose hydrophilicity values are within +/−2 is preferred, those which are within +/−1 are particularly preferred, and those within +/−0.5 are even more particularly preferred. Hence, in reference to hydrophilicity, arginine, lysine, aspartic acid, and glutamic acid are defined herein as biologically functional equivalents, particularly in water soluble amino acid polymers.

In certain embodiments, pseudo-poly(amino acids) may also be used in the present invention. Pseudo-poly(amino acids) differ from the poly(amino acids) described above in that dipeptide monomers are covalently bound through other than the normal peptide linkages. Pseudo-poly(amino acids) suitable for use in accordance with the present invention are those, for example in Kohn, J. and Langer, R., Polymerization Reactions Involving the Side Chains of α-L-Amino Acids, J. Amer. Chem. Soc., 109, 917 (1987) and Pulapura, S. and Kohn, J., Biomaterials Based on “Pseudo”-Poly(Amino Acids): A Study of Tyrosine Derived Polyiminocarbonates, J. Polymer Preprints, 31, 23 (1990), each of which are incorporated herein by reference. The pseudo-poly(amino acids) can be used alone or in combination with the mixtures of classical poly(amino acids) and pseudo-poly(amino acids) in accordance with the invention.

The manufacture of a poly(amino acid) polymer is well-known to the person of ordinary skill in the art. For example, a homopolymer of glutamic acid may be prepared in a two-step process, in which (i) glutamic acid is treated with phosgene or an equivalent reagent, e.g., diphosgene, at a temperature of from 15° C. to 70° C. to form an N-carboxyanhydride (NCA), and (ii) ring-opening polymerization of the N-carboxyanhydride is effected with a base to yield poly(glutamic acid). Suitable bases include alkoxides, e.g., alkali metal alkoxides such as sodium mothoxide, organometallic compounds and primary, secondary or tertiary amines, for example butylamine or triethylamine. See U.S. Pat. No. 5,470,510. There are numerous methods for chemically synthesizing poly(amino acids). See, e.g., Hayashi et al., Biopolymers, 29: 549, 1990; Hayashi et al., J. Appl. Polym. Sci., 43: 2223, 1991; and Hayashi et al., Polym. J., 5: 481,1993).

In certain aspects, the amino acid polymers of the present invention may be produced recombinantly by any means suitable, such as by utilizing transformed E. coli to produce the same. For example, limited bacterial production of poly (glutamic acid) is described, for example in EP-A-410, 638 (Takeda). Bacterial synthetic processes will commonly yield poly (L-glutamic acid), although bacteria are known that will provide the D-form.

Antigenic Compounds

The present invention provides immunogenic compositions that enhance the immunogenicity of an antigenic compound. As used herein, the term “antigenic compound” is meant to refer to any substance that can be recognized by an antibody. The “antigenic compound” includes any antigen or hapten. A “hapten” is defined as an antigenic molecule that is substantially non-immunogenic by itself but can become immunogenic when associated with a larger molecule, sometimes referred to as a carrier.

Antigenic compounds of the invention thus include any antigen or hapten to which the generation or enhancement of an immune response is desired. In certain embodiments, antigenic compounds are organisms or moieties associated with disease, infection, such as, for example, a microorganism or disease-associated antigen, or epitope derived therefrom.

In one embodiment, an immunogenic composition of the invention comprises a cancer- or tumor-related antigen or hapten. As used herein, a cancer-related antigen or hapten is any antigen or hapten associated with the presence of a tumor in a patient. In certain embodiments, cancer antigens are recognized by antibodies or T cells of the patient. Cancer-related antigens include all known and unknown human tumor antigens. Examples of known human tumor antigens include, but are not limited to, carcinoembryonic antigen, carcinoembryonic antigen peptide-1 (CAP-1), α-fetoprotein, alkaline phosphatase isoenzyme, prostate-specific antigen, beta subunit of choriogonadotropic hormone, calcitonin, Bence-Jones proteins, aspartyl β-hydroxylase, NY-ESO-1, 707 alanine proline, adenocarcinoma antigen (ART-4), B antigen (BAGE), β-catenin, m-catenin, Bcr-abl, CTL-recognized antigen on melanoma (CAMEL), caspase-8, CDC27, CDK4, cancer/testis antigen, cyclophilin B, differentiation antigens melanoma (DAM-6 and DAM-10), elongation factor 2, Ets, glycoprotein 250, G antigen, N-acetylglucosaminyltransferase V, glycoprotein 100 kD, helicose antigen, human epidermal receptor-2/neurological (HER2-neu), HLA-A2 R170I, human papilloma virus E7, heat shock protein 70—2 mutated, human signet ring tumor—2, human telomerase reverse transcriptase, intestinal carboxyl esterase, L antigen, low density lipid receptor/GDP-L-fucose:-D-galactosidase 2-L-fucosyltransferase, melanoma antigen, Melanoma antigen A, melanocortin 1 receptor, myosin mutated, mucin 1, melanoma ubiquitous mutated 1, 2, 3, NA cDNA clone of patient M88, New York—esophageous 1, protein 15, minor bcr-abl, promyelocytic leukaemia/retinoic acid receptor (Pml/RAR), preferentially expressed antigen of melanoma, prostate-specific antigen, prostate-specific membrane antigen, renal antigen, renal ubiquitous 1 or 2, sarcoma antigen, squamous antigen rejecting tumor 1 or 3, translocation Ets-family leukemia/acute myeloid leukemia 1 (TEL/AML1), triosephosphate isomerase mutated, gp75, tyrosinase related protein 2, TRP-2/intron, and Wilms' tumor gene. A variety of tumor antigens are described in Renkvist, N. et al., A Listing of Human Tumor Antigens Recognized by T Cells, available at http://www.istitutotumori.mi.it/menurisorse/listing/pdf. Additional tumor antigens are described in the Institute for Cancer Research database, available at http://www.licr.org/SEREX.htm, and include all tumor antigens identified according to serological identification and cloning methods described in Sahin, U. et al., Curr. Opin. Immunol. 9:709-716 (1997) and Chen, Y. T. et al., Principles and Practice of Biologic Therapy of Cancer, 3rd Ed., S. A. Rosenberg, ed., Lippincott, Williams, and Wilkins, Philadelphia, Pa., pp. 557-570 (2000), and references cited therein.

In one embodiment, an immunogenic composition of the invention comprises a microbe, such as, for example, a virus, bacteria, fungi, mycoplasma, or protozoa, or an antigenic fragment, fraction, extract, peptide, or moiety derived from a microbe. In certain embodiments, of the invention, the microbe is an infectious agent associated with a disease or pathologic condition.

In certain embodiments, antigens or haptens are derived from infectious agents associated with human diseases, including, but not limited to, cutaneous anthrax, inhalation anthrax, gastrointestinal anthrax, nosocomical Group A streptococcal infections, Group B streptococcal disease, meningococcal disease, blastomycocis, streptococcus pneumonia, botulism, Brainerd Diarrhea, brucellosis, pneumonic plague, AIDS, candidiasis (including oropharyngeal, invasive, and genital), drug-resistant Streptococcus pneumoniae disease, E. coli infections, Glanders, Hansen's disease (Leprosy), cholera, tularemia, histoplasmosis, legionellosis, leptospirosis, listeriosis, meliodosis, mycobacterium avium complex, mycoplasma pneumonia, tuberculosis, peptic ulcer disease, nocardiosis, chlamydia pneumonia, psittacosis, salmonellosis, shigellosis, sporotrichosis, strep throat, toxic shock syndrome, trachoma, traveler's diarrhea, typhoid fever, ulcer disease, and waterborne disease.

The antigens or haptens may be derived from infectious agents associated with human tumors or malignancies, such as, for example, Epstein-Barr virus, Helicobacter pylori, Hepatitis B virus, Hepatitis C virus, Human heresvirus-8, Human immunodeficiency virus, Human papillomavirus, Human T cell leukemia virus, liver flukes, or Schistosoma haematobium.

In certain aspects, the present invention provides that antigenic compounds include, but are not limited to, synthetic or naturally derived proteins and peptides; carbohydrates including, but not limited to, polysaccharides; lipids; and antigens isolated from biological sources such as, for example microbes, viruses, or parasites, and subunits or extracts therefrom; or any combination thereof. Exemplary antigens include Streptococcus pneumoniae, S. typhi VI carbohydrate, Hemophilus influenzae (type B), Acellular B. pertussis, Neisseria meningiditis (A,C), H. influenzae (type B, Hib), Clostridium tetani (tetanus), and Corynebacterium diphtheriae (diphtheria), and subunits or moieties derived therefrom.

In certain embodiments, the immunogenic composition according to the present invention may, for example, comprise at least one antigenic compound selected from the group consisting of: (A) live, heat killed, or chemically attenuated viruses, bacteria, mycoplasmas, fungi, and protozoa; (B) fragments, extracts, subunits, metabolites and recombinant constructs of (A); (C) fragments, subunits, metabolites and recombinant constructs of mammalian proteins, glycoproteins, lipids, and other epitopes; (D) tumor-specific antigens; and (E) nucleic acid molecules (e.g., RNA and DNA).

In certain embodiments, the antigenic compound of the present invention comprises a peptide, polypeptide, or protein. Examples of an antigen include, but are not limited to an allergen, a viral antigen, a bacterial antigen such as a bacterial DNA, a protozoan antigen, a tumor antigen, a fungal antigen; an infectious disease antigen or a mixture thereof. Specifically, for example, a tumor antigen can be Her-2/neu protein, protein fragments or peptides, PSA, PSM, mammaglobin, prolactin inducing protein (PIP), p21 or p53, an infectious disease antigen can be hepatitis B surface antigens, hepatitis C antigens, malaria antigens, TB antigens, chlamydia antigens, Herpes antigens, flu antigens, HIV antigens, EBV antigens, papilloma antigens, and H. pylon antigens.

Antigenic compounds, including antigenic fragments of a protein, can readily be determined by standard means of determining antigenicity of substances.

In certain aspects, the present invention provides an antigen such as a virus, a microorganism, more particularly a bacterium or parasite, or a compound comprising a peptide chain. Such a compound may include a protein or a glycoprotein, especially a protein or glycoprotein obtained from a microorganism, a synthetic peptide or a protein or a peptide obtained by genetic engineering. The virus and microorganism may be totally inactivated or live and attenuated.

A composition according to the invention may also comprise an in vivo generator of an antigenic compound comprising an amino acid sequence, that is to say the in vivo generator capable of expressing the antigenic compound in the host organism into which the in vivo generator has been introduced. The antigenic compound comprising the amino acid sequence may be a protein, a peptide or a glycoprotein, for example. The in vivo generators are generally obtained by genetic engineering processes. More particularly, the in vivo generators may comprise living microorganisms, generally a virus, acting as a recombinant vector, into which is inserted a nucleotide sequence, in particular an exogenous gene. In this regard, reference may be made to the article by M. Eloit et al., Journal of Virology 71, 2925-2431, 1990, to International Application WO-A-91.00107 or to International Application WO-A-94/16681.

In certain embodiments, HSV Glycoprotein D (gD) or derivatives thereof is a useful antigen. It is located on the viral membrane, and is also found in the cytoplasm of infected cells (Eisenberg et al., J of Virol 35: 428-435, 1980). It comprises 393 amino acids including a signal peptide and has a molecular weight of approximately 60 kD. Of all the HSV envelope glycoproteins this is probably the best characterized (Cohen et al., J. Virology 60: 157-166). In vivo, it is known to play a central role in viral attachment to cell membranes. Moreover, glycoprotein D has been shown to be able to elicit neutralizing antibodies in vivo and protect animals from lethal challenge. A truncated form of the gD molecule is devoid of the C terminal anchor region and can be produced in mammalian cells as a soluble protein which is exported into the cell culture supernatant. Such soluble forms of gD are preferred. The production of truncated forms of gD is described in EP 0 139 417. Preferably the gD is derived from HSV-2. An embodiment of the invention is a truncated HSV-2 glycoprotein D of 308 amino acids which comprises amino acids 1 through 306 naturally occurring glycoprotein with the addition Asparagine and Glutamine at the C terminal end of the truncated protein devoid of its membrane anchor region. This form of the protein includes the signal peptide which is cleaved to allow for the mature soluble 283 amino acid protein to be secreted from a host cell.

In another aspect of the invention, Hepatitis B surface antigen is a useful antigen. As used herein the expression “Hepatitis B surface antigen” or “HbsAg” includes any HBsAg antigen or fragment thereof displaying the antigenicity of HBV surface antigen, including the 226 amino acid sequence of the HBsAg antigen (see Tiollais et al., Nature, 317: 489,1985, and references therein). HBsAg as herein described may, if desired, contain all or part of a pre-S sequence as described in the above references and in EP-A-0 278 940. In particular, the HBsAg may comprise a polypeptide comprising an amino acid sequence comprising residues 12-52 followed by residues 133-145 followed by residues 175-400 of the L-protein of HBsAg relative to the open reading frame on a Hepatitis B virus of ad serotype (this polypeptide is referred to as L*; see EP 0 414 374). HBsAg within the scope of the invention may also include the pre-S1-preS2-S polypeptide described in EP 0 198 474 (Endotronics) or close analogues thereof such as those described in EP 0 304 578 (Mc Cormick and Jones). HBsAg as herein described can also refer to mutants, for example the “escape mutant” described in WO 91/14703 or European Patent Application Number 0 511 855A1, especially HBsAg wherein the amino acid substitution at position 145 is to arginine from glycine.

In another embodiment, a useful antigen is an RSV antigen, in particular an F/G antigen. U.S. Pat. No. 5,194,595 (Upjohn) describes chimeric glycoproteins containing immunogenic segments of the F and G glycoproteins of RSV and suggests that such proteins can be expressed from a variety of systems including bacterial, yeast, mammalian (e.g., CHO cells) and insect cells (using for example a baculovirus). Wathen et al., (J. Gen. Virol. 70: 2625-2635, 1989) describe a particular RSV FG chimeric glycoprotein expressed using a baculovirus vector consisting of amino acids 1-489 of the F protein linked to amino acids 97-279 of the G protein.

In one other embodiment of the invention, an antigen may be derived from an HIV virus, such as HIV-1. Potential HIV-1 antigens include, for example, HIV-1 polypeptides and fragments or peptides thereof, including surface proteins; live, attenuated HIV-1 virus; whole, killed HIV-1 virus, naked DNA comprising an HIV-1 gene or fragment thereof; bacterial or non-HIV-1 viral vectors engineered to carry an HIV-1 gene or fragment thereof; pseudovirions or other non-replicating HIV-1-like particles capable of presenting HIV-1 surface or internal proteins; and replicons or other non-HIV-1 viruses with limited ability to replicate that carry an HIV-1 gene or fragment thereof. Any known or identified HIV antigen may be used according to the present invention, including, for example, recombinant HIV-1 p24 antigen, recombinant HIV-1 gp41 antigen, and recombinant HIV-2 gp36 antigen, each commercially available from The Binding Site, Inc., San Diego.

In certain embodiments, the antigenic compounds include allergens that elicit an allergic response. These include, for example, proteins found in food, such as strawberries, peanuts, milk proteins, egg whites, etc. Other allergens of interest include various airborne antigens, such as grass pollens, animal danders, house mite feces, etc. Molecularly cloned allergens include Dermatophagoides pteryonyssinus (Der P1); Lol pl-V from rye grass pollen; a number of insect venoms, including venom from jumper ant Myrmecia pilosula; Apis millifera bee venum phospholipase A2 (PLA₂) and antigen 5S; phospholipases from the yellow jacket Vespula maculifrons and the white faced hornet Dolichovespula maculata; a large number of pollen proteins, including birch pollen, ragweed pollen, Parol (the major allergen of Parietaria officinalis) and the cross-reactive allergen Parjl (from Parietaria judaica), and other atmospheric pollens including Olea europaea, Artemisia sp., gramineae, etc. Other allergens of interest are those responsible for allergic dermatitis caused by blood sucking arthropods, e.g., Diptera, including mosquitoes (Anopheles sp., Aedes sp., Cuffseta sp., Culex sp.); flies (Phlebotomus sp., Culicoides sp.) particularly black flies, deer flies and biting midges; ticks (Dermacenter sp., Omithodoros sp., Otobius sp.); fleas, e.g., the order Siphonaptera, including the genera Xenopsylla, Pulex and Ctenocephalides. Reviews of molecularly cloned allergens include Chapman et al. (1997) Allergy 52(4):374-9 “Recombinant mite allergens”; King (1996) Toxicon. 34(11-12): 1455-88, “Immunochemical studies of stinging insect venom allergens”; Becker et al. (1995) Int Arch Allergy Immunol. 107(1-3):242-4, “Molecular characterization of timothy grass pollen group V allergens”; and Scheiner et al. (1994) Arb Paul Ehrlich Inst Bundesamt Sera Impfstoffe Frankf A M. (87):221-32, “Molecular and functional characterization of allergens: basic and practical aspects”.

In other embodiments, antigens and haptens include those associated with immune disorders, including autoimmune diseases, such as autoimmune thyroid disease, including Graves' disease and Hashimoto's thyroiditis, rheumatoid arthritis, systemic lupus erythematosus (SLE), Sjogrens syndrome, immune thrombocytopenic purpura (ITP), multiple sclerosis (MS), myasthenia gravis (MG), psoriasis, scleroderma, and inflammatory bowel disease (IBD), including Crohn's disease, and ulcerative colitis, for example. In patients with autoimmune disease, inflammatory cells (e.g., white blood cells, such as T cells), which normally defend the body against antigens (bacteria, viruses, etc.) are unable to distinguish between foreign substances and the body's own tissue. As a result, the inflammatory cells attack healthy tissue, causing chronic inflammation. These self-destructive immune responses may occur in such tissues as the joint surfaces (rheumatoid arthritis), the thyroid gland (Grave's disease), the central nervous system (multiple sclerosis), and the intestine (IBD), for example. In each case, the inflammatory cells target specific proteins, which are normally found in the tissues of these organs. Such proteins are, therefore, known as “self-antigens” or “autoantigens.” Studies indicate that it is possible to inhibit the autoimmune response in a patient suffering from an autoimmune disease by treating the patient with autoantigen(s) that play a role in their disease. Such treatment effectively restores the patient's ability to tolerate these proteins, a process known as tolerance. Accordingly, the invention also provides a method of inducing tolerance to an antigen or hapten by introducing an antigen or hapten to a patient suffering from an immune response directed against the antigen or hapten.

Antigens and haptens may be produced by methods known in the art or may be purchased from commercial sources. Antigens and haptens may be derived from natural sources or may be synthesized. For example, U.S. Pat. Nos. 4,434,157, 4,406,885, 4,264,587, 4,117,112, 4,034,081, 3,996,907, incorporated herein by reference, describe methods for preparing antigens for feline leukemia virus vaccines. Other antigens may similarly be prepared. As noted above, antigens within the scope of this invention include whole inactivated virus particles, isolated virus proteins and protein subunits, whole cells and bacteria, cell membrane and cell wall proteins, and the like. Compositions of the invention may be used to immunize birds and mammals against diseases and infection, including without limitation cholera, diptheria, tetanus, pertussis, influenza, measles, meningitis, mumps, plague, poliomyelitis, rabies, Rocky Mountain spotted fever, rubella, smallpox, typhoid, typhus, feline leukemia virus, and yellow fever.

Examples of suitable antigens that may be used in the present invention include any antigen that is used as a vaccine for a single disease (“single antigen”) or two or more diseases simultaneously (“mixed antigen”). The mixed antigen may be a mixture of two or more antigens, or an antigen that has antigenicities for two or more diseases simultaneously, e.g., a recombinant protein. As an antigen, there may be used an entire organism, e.g., a viral or bacterial whole cell, or a part of the organism, e.g., a certain protein having an antigenicity.

In one embodiment, an immunogenic composition of the present invention can be monovalent or multivalent (i.e., it can protect an animal from one or more other infectious agents). Antigens may be derived from any source, including, but not limited to, one or more other infectious agents, such as, but not limited to: viruses, e.g., adenoviruses, caliciviruses, coronaviruses, distemper viruses, hepatitis viruses (e.g., hepatitis A, B, C, and D viruses), herpesviruses, immunodeficiency viruses, infectious peritonitis viruses, leukemia viruses, oncogenic viruses, papilloma viruses, parainfluenza viruses, parvoviruses, rabies viruses, and reoviruses, as well as other cancer-causing or cancer-related viruses; bacteria, e.g., Actinomyces, Bacillus, Bacteroides, Bordetella, Bartonella, Borrelia, Brucella, Campylobacter, Capnocytophaga, Clostridium, Corynebacterium, Coxiella, Dermatophilus, Enterococcus, Elirlichia, Escherichia, Francisella, Fusobacterium, Haemobartonella, Helicobacter, B. henselae, Klebsiella, L-form bacteria, Leptospira, Listeria, Mycobacteria, Mycoplasma, Neorickettsia, Nocardia, Pasteurella, Peptococcus, Peptostreptococcus, Proteus, Pseudomonas, Rickettsia, Rochalimaea, Salmonella, Shigella, Staphylococcus, Streptococcus, and Yersinia; fungi and fungal-related microorganisms, e.g., Absidia, Acremonium, Alternaria, Aspergillus, Basidiobolus, Bipolaris, Blastomyces, Candida, Chlamydia, Coccidioides, Conidiobolus, Cryptococcus, Curvalaria, Epidermophyton, Exophiala, Geotrichum, Histoplasma, Madurella, Malassezia, Microsporum, Monilella, Mortierella, Mucor, Paecilomyces, Penicillium, Phialemonium, Phialophora, Prototheca, Pseudallescheria, Pseudomnicrodochium, Pythium, Rhinosporidium, Rhiizopus, Scolecobasidium, Sporotlirix, Stempylium, Trichophyton, Trichosporon, and Xylohypha; parasites, e.g., Babesia, Balantidium, Besnoitia, C. typtosporidium, Eimeria, Encephalitozoon, Entamoeba, Giardia, Hammondia, Hepatozoon, Isospora, Leishmania, Micro sporidia, Neospora, Nosema, Pentatrichomonas, Plasmodium, Pneumocystis, Sarcocystis, Schistosoma, Theileria, Toxoplasma, and Trypanosoma, and helminth parasites, e.g., Acanthocheilonema, Aelurostrongylus, Ancylostoma, Angiostrongylus, Ascaris, Brugia, Bunostomum, Capillaria, Chabertia, Cooperia, Crenosorna, Dictyocaulus, Dioctophyme, Dipetalonema, Diphyllobothrium, Diplydium, Dirofilaria, Dracunculus, Enterobius, Filaroides, Haemonchus, Lagochilascaris, Loa, Mansonella, Muellerius, Naiophyetus, Necator, Neniatodirus, Oesophagostomum, Onchocerca, Opisthorchis, Ostertagia, Parafilaria, Paragonimus, Parascaris, Physaloptera, Protostrongylus, Setaria, Spirocerca, Spirometra, Stephanofilaria, Strongyloides, Strongylus, Thelazia, Toxascaris, Toxocara, Trichinella, Trichostrongylus, Trichuris. Uncinaria, and Wuchereria.

In certain embodiments, the antigen or hapten is a peptide or polypeptide. Such antigens or haptens may be purified from a natural source or they may be synthesized or recombinantly produced, according to a wide variety of methods known and available in the art. In certain embodiments, the antigenic compound is a polypeptide that encoded by a polynucleic acid sequence, and the carrier is a poly(amino acid) polymer encoded by a polynucleic acid sequence. Thus, the antigenic compound-carrier conjugate may be encoded by one polynucleotide construct. Therefore, the antigenic compound-carrier conjugate can be produced by recombinant technologies that are well known to the person of ordinary skill in the art, including bacterial and yeast recombinant expression systems, for example. One advantage associated with using recombinant antigen instead of heat-killed or denatured microbes (e.g., bacteria and viruses) is that it avoids the possibility that a small fraction of the microbe has not been killed or inactivated and the associated threat of infection.

Conjugation

The carriers (e.g., polymers) of the present invention may be associated with the antigenic compound (e.g., antigen or hapten) in any manner known or available to those skilled in the art. For example, the carrier can be associated with the antigenic compound through a covalent bond, including peptide bonds, for example, or through charge-charge interactions, vander wahl forces, and the like. With respect to covalent bonds, such may be generated synthetically or by virtue of a genetic fusion that produces the polymer and antigenic compound recombinantly. Exemplary methods of conjugating a carrier of the invention to an antigen/hapten are described, for example, in U.S. Pat. Nos. 5,977,163 and 6,262,107, U.S. Patent Application Serial No. 60/013,184, Ser. Nos. 09/050,662, 09/530,601, No. 60,159,135, Ser. No. 09/686,627, No. 60/190,429, Ser. No. 09/810,345, No. 60,277,705, and Ser. No. 09/956,237; and PCT Publication Nos. WO 99/49901, WO 97/33552, WO 01/26693, and WO 01/70275, which are incorporated by reference herein.

Those of ordinary skill in the art will readily understand that the carrier (e.g., polymer) and the antigenic compound may be conjugated or associated directly or through a secondary molecule such as a linker or spacer. Preferred linkers include those that are relatively stable to hydrolysis in the circulation. Exemplary linkers include amino acids, hydroxyacidsdiols, aminothiols, hydroxythiols, aminoalcohols, and combinations of these. In addition, the antigen/hapten may require modification prior to conjugation, e.g., the introduction of a new functional group, the modification of a preexisting functional group or the attachment of a spacer molecule.

Chemical coupling may be achieved using commercially available homo- or hetero-bifunctional cross-linking compounds, according to methods known and available in the art, such as those described, for example, in Hermanson, Greg T., Bioconjugate Techniques, Academic Press, Inc., 1995, and Wong, Shan S., Chemistry of Protein Conjugation and Cross-linking, CRC Press, 1991, both of which are hereby incorporated by reference.

Additional examples of how carriers may be linked to antigenic compounds or linkers are described in Hoffman et al., Biol. Chem. 370:575-582, 1989; Wiesmuller et al., Vaccine, 7:29-33, 1989; Wiesmuller et al., Int. J. Peptide Protein Res., 40:255-260,1992; Defourt et al., Proc. Natl. Acad. Sci. 89:3879-3883, 1992; Tohokuni et al., J. Am. Chem. Soc., 116:395-396, 1994; Reichel, Chem. Commun., 2087-2088, 1997; Kamitakahara, Angew. Chem. Int. Ed. 37:1524-1528, 1998; Dullenkopf et al., Chem. Eur. J., 5:2432-2438,1999; all of which are hereby incorporated by reference.

In certain embodiments, a polymer carrier of the invention is conjugated to an antigen or hapten by chemical conjugation, as described in U.S. Pat. No. 5,977,163. In this method, polyglutamic acid conjugates are prepared as a sodium salt, dialyzed to remove low molecular weight contaminants and excess salts, and then lyophilized.

In another embodiment, a polymer carrier of the invention is conjugated to an antigen or hapten by chemical conjugation, essentially as described in the published PCT application, WO 01/26693 A2. According to this method, a polyglutamic acid polymer is covalently bonded to an antigen or epitope by a direct linkage between a carboxylic acid residue of the polyglutamic acid and a functional group of the antigen/hapten, or by an indirect linkage via one or more bifunctional groups. An antigen or hapten can be linked to a polymer or linker by any linking method available in the art and according to methods well known to those skilled in the art, including those found, for example, in March, J., Advanced Organic Chemistry, Wiley Interscience, 4th ed., 1992.

In one embodiment, a polyglutamate carrier is coupled to an antigen or hapten according to a method comprising the following steps:

(a) providing a protonated form of a polyglutamic acid polymer and an antigen/hapten for conjugation thereto;

(b) covalently linking said antigen/hapten to said polyglutamic acid polymer in an inert organic solvent to form a polyglutamic acid-antigen/hapten conjugate;

(c) precipitating said polyglutamic acid-antigen/hapten conjugate from solution by addition of an excess volume of aqueous salt solution; and

(d) collecting said conjugate as a protonated solid.

The protonated form of the polyglutamic acid polymer in step (a) is obtained by acidifying a solution containing the salt of the polyglutamic acid to be used as a starting material, and converting the salt to its acid form. After separating the solid by centrifugation, the solid is washed with water. The polyglutamic acid is then dried, preferably by lyophilization and preferably to a constant weight comprising between about 2% to about 21% water, between about 7% to about 21% water, or between 7% and 21% water, prior to conjugation to a desired antigen/hapten (step (b)).

Compositions of the invention may be produced in whole, or in part, using recombinant DNA technology, as is widely known and available in the art. For example, a carrier or an antigen, or both, may be produced by recombinant means and thereafter associated or conjugated. Alternatively, a single polypeptide, for example, comprising both the carrier and the antigen or hapten may be produced as a fusion protein. Methods of constructing recombinant expression vectors are known in the art, as are methods of expressing recombinant polypeptides in a variety of organisms, such as, for example, bacteria and yeast. Such methods are described, for example, in U.S. Patent Application Serial No. 60/277,705.

The amount of antigenic compound, including antigens or haptens, conjugated per polymer can vary. At the lower end, the antigenic compound-polymer conjugate may comprise from about 1%, about 2%, about 3%, about 4%, about 5%, about 6%, about 7%, about 8%, about 9%, or about 10%, about 11%, about 12%, about 13%, about 14%, about 15%, about 16%, about 17%, about 18%, about 19%, about 20%, about 21% about 22%, about 23%, about 24%, to about 25% (w/v) antigenic compounds including antigens relative to the mass of the conjugate. At the high end, the antigenic compound-polymer conjugate may comprise from about 26%, about 27%, about 28%, about 29%, about 30%, about 31% about 32%, about 33%, about 34%, about 35%, about 36%, about 37%, about 38%, about 39%, about 40%, to about 50% or more (w/w) antigenic compound including antigens relative to the mass of the conjugate.

In certain other aspects of the invention, the number of molecules of antigenic compound including antigens conjugated per molecule of polymer can vary. At the lower end, the antigenic compound-polymer conjugate may comprise from about 1, about 2, about 3, about 4, about 5, about 6, about 7, about 8, about 9, about 10, about 11, about 12, about 13, about 14, about 15, about 16, about 17, about 18, about 19, to about 20 or more molecules of the antigenic compound, including antigens per molecule of polymer. At the higher end, the antigenic compound-polymer conjugate may comprise from about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60 about 61, about 62, about 63, about 64, about 65, about 66, about 67, about 68, about 69, about 70, about 71, about 72, about 73, about 74, to about 75 or more molecules or more of antigenic compound, including antigens per molecule of water soluble polymer.

It should be recognized that a carrier may be associated with one or more discrete or overlapping sites on an antigen or hapten. Similarly, in certain embodiments, an antigen or hapten may be associated with one or more discrete sites on a carrier. Accordingly, in certain embodiments, compositions of the invention include carriers associated with antigens through different sites on an antigen, as well as antigens associated with carriers through different sites on the carrier. Different linkers may be used to direct association through different sites, or a single linker may be used, depending on the particular functional groups present at each site. In certain embodiments, the invention includes a composition comprising a mixture of one or more carriers associated with one or more antigens through one or more different or overlapping sites on an antigen or carrier. The association or conjugation of an antigen to a carrier through different sites on the antigen or carrier allows different configurations suitable for displaying or presenting multiple epitopes of an antigen, thereby facilitating the generation of an immune response directed against multiple epitopes on an antigen.

Adjuvants

In certain embodiments, an immunogenic composition optionally comprises one or more adjuvants. As used herein, the term “adjuvant” refers to any substance or mixture of substances that increases or diversifies the immune response to an antigenic compound. Adjuvants provided below are merely exemplary. In fact, any adjuvant may be used in the immunogenic composition of the present invention as long as the adjuvant satisfies the requisite characteristics that are necessary for practicing the present invention. As indicated above, the carrier of the compositions of the present invention itself may act as an adjuvant.

The use of adjuvants in therapeutic compositions of the vaccine type is well known. The main objective of these adjuvants is to allow an increase in the immune response. These adjuvants are diverse in nature. They may, for example, consist of liposomes, oily phases, including, for example, the Freund type of adjuvants, such as complete Freund's adjuvant and incomplete Freund's adjuvant. Such adjuvants are generally used in the form of an emulsion with an aqueous phase, or, more commonly, may consist of water-insoluble inorganic salts. These inorganic salts may consist, for example, of aluminum hydroxide, zinc sulfate, colloidal iron hydroxide, calcium phosphate or calcium chloride. Aluminum hydroxide (Al(OH)₃) is a commonly used adjuvant. These adjuvants are described, in particular, in Gupta et al. Vaccine, 11: 993-306,1993, and in Arnon, R. (Ed.) Synthetic Vaccines 1:83-92, CRC Press, Inc., Boca Raton, Fla., 1987.

In certain embodiments, the composition of the present invention comprises an antigen that is employed in a mixture with the adjuvant compounds. In other formulations of the adjuvant of the present invention, it may be useful in some applications to employ an antigen covalently linked to an amino, carboxyl, hydroxyl and/or phosphate moiety of the adjuvant compounds of the invention. The specific formulation of compositions of the present invention may thus be carried out in any suitable manner which will render the adjuvant bioavailable, safe and effective in the subject to whom the formulation is administered.

The resulting compositions, including (i) an antigenic compound, (ii) a carrier including a polymer of the present invention, and, optionally, (iii) an adjuvant compound, are usefully employed to induce an immunological response in an animal, by administering to such animal the compositions, in an amount sufficient to produce an antibody response in such animal.

In certain embodiments, the adjuvant may be selected from the group including, but not limited to, cytokines, chemokines, growth factors, angiogenic factors, apoptosis inhibitors, and combinations thereof. When a cytokine is chosen as an adjuvant or antigen, the cytokine may comprise IL-1, IL-2, IL-6, IL-12, IL-15, IL-18, IFN-a, IFN-a, GM-CSF, Flt31, or a mixture thereof. These cytokines could be administered either as soluble or non-soluble entities with an antigen or in microsphere formulations or encapsulated antigens or microspheres.

In certain embodiments, combinations of cytokines are also contemplated for use in accordance with the methods of the present invention. Additionally, a particularly contemplated embodiment comprises the use of IL-12 and IL-18 in combination as a mucosal adjuvant in accordance with the methods of the present invention. When cytokines are used in combination, contemplated dosage ranges comprise about 0.3 μg/ml to about 50 μg/ml, with respect to each cytokine. Portions of cytokines, or muteins or mimics of cytokines (or combinations thereof), having adjuvant activity or other biological activity can also be used in the methods of the present invention.

Other examples of substantially non-toxic, biologically active mucosal adjuvants of the present invention include hormones, growth factors, or biologically active portions thereof. Such hormones, growth factors, or biologically active portions thereof can be of human, bovine, porcine, ovine, canine, feline, equine, or avian origin, for example, and can be tumor necrosis factor (TNF), prolactin, epidermal growth factor (EGF), granulocyte colony stimulating factor (GCSF), insulin-like growth factor (IGF-1), somatotropin (growth hormone) or insulin, or any other hormone or growth factor whose receptor is expressed on cells of the immune system.

Cytokines, chemokines, growth factors, angiogenic factors, apoptosis inhibitors and hormones can be obtained from any suitable source or produced by recombinant DNA methodology. For example, the genes encoding several human interleukins have been cloned and expressed in a variety of host systems, permitting the production of large quantities of pure human interleukin. Further, certain T lymphocyte lines produce high levels of interleukin, thus providing a source of the cytokine.

Other examples of adjuvants that are useful in the present invention include but are not limited to plasmid DNA or bacterial agents. An adjuvant can also include, for example, an immunomodulator. An immunomodulator could upregulate co-stimulatory molecules such as B7 or CTLA-4 or it could enhance Th1 type responses. Molecules that enhance a Th1 type response in vivo could be administered with antigen containing microspheres to enhance T-cell responses preferentially. An example of such a molecule is LeIF, a leishmania derived protein that has been shown to induce a Th1 response. Furthermore, a nucleic acid encoding a co-stimulatory molecule can be administered to provide the co-stimulatory molecule.

Additional adjuvants include any compound described in Chapter 7 (pp 141-227) of “Vaccine Design, The Subunit and Adjuvant Approach” (eds. Powell, M. F. and Newman, M. J.) Pharmaceutical Biotechnology, Volume 6, Plenum Press (New York). Examples from this compendium include Muramyl Dipeptide (MDP) and Montanide 720. Molecules such as Poly Inosine:Cytosine (Poly I:C) or plasmid DNA containing CpG motifs can also be administered as adjuvants in combination with antigens encapsulated in microparticles. In another example, the adjuvant is an agent that facilitates entry of the antigenic compound into the cytoplasm of a cell such as listeriolysin, streptolysin or a mixture thereof.

In certain embodiments, the immunogenic composition of the present invention may comprise an oily adjuvant. The oily adjuvant may be a mineral oil, a non-mineral oil or a mixture of a mineral oil and a non-mineral oil. Mineral oils may be natural or synthetic. Non-mineral oils may be of plant, animal or synthetic origin. The non-mineral oils are advantageously metabolizable. All these oils are devoid of toxic effects with regard to the host organism into which the composition of the invention is administered. They are preferably liquid at the storage temperature (about +4° C.) or at least make it possible to give emulsions which are liquid at this temperature. An advantageous mineral oil according to the invention may include an oil comprising a linear carbon chain having a number of carbon atoms preferably greater than 16, and free of aromatic compounds. Such oils may, for example, be those marketed under the name “MARCOL 52” (produced by Esso France) or “DRAKEOL 6VR” (produced by Penreco USA).

Examples of synthetic non-mineral oils which may be mentioned are polyisobutenes, polyisopropenes, esters of alcohols and fatty acids, such as, for example, ethyl oleate and isopropyl myristate, mono-, di- or triglycerides, propylene glycol esters, partial glycerides such as corn oil glycerides, for instance those marketed by the company SEPPIC under the name LANOL®, maisin and oleyl oleate. Among the plant oils which may be mentioned are unsaturated oils rich in oleic acid which are biodegradable, for example groundnut oil, olive oil, sesame oil, soya oil or wheatgerm oil.

The animal oils may include, in particular, squalene, squalane or spermaceti oil.

The oily adjuvant may also include a self-emulsifiable oil, that is to say an oily preparation capable of forming a stable emulsion with an aqueous phase, with virtually no energy input, for example by dispersion in the aqueous phase by slow mechanical stirring. In this respect, self-emulsifiable oils such as those known in the European Pharmacopoeia under the names Labrafil and Simulsol may be mentioned. These oils are polyglycolyzed glycerides.

The immunogenic composition of the present invention may comprise a non-toxic double mutant form of pertussis toxin as adjuvants. The non-toxic double mutant is preferably one in which the glutamic acid 129 amino acid in the S₁ sub-unit has been substituted by glycine and the arginine 9 amino acid has been substituted by lysine. See U.S. Pat. Appl. 20010018056.

In certain embodiments, the immunogenic composition of the present invention may comprise saponins as adjuvants. As a class, saponins are described in Lacaille-Dubois and Wagner, Phytomedicine 2: 363-386, 1996. Saponins are steroid or triterpene glycosides widely distributed in the plant and marine animal kingdoms. Saponins are noted for forming colloidal solutions in water that foam on shaking, and for precipitating cholesterol. When saponins contact cell membranes, they create pore-like structures in the membrane that cause the membrane to burst. Haemolysis of erythrocytes is an example of this phenomenon, which is a property of certain, but not all, saponins.

Saponins are known as adjuvants in vaccines for systemic administration. The adjuvant and haemolytic activity of individual saponins has been extensively studied in the art (Lacaille-Dubois and Wagner, supra). For example, Quil A (derived from the bark of the South American tree Quillaja Saponaria Molina), and fractions thereof, are described in U.S. Pat. No. 5,057,540 and “Saponins as vaccine adjuvants”, Kensil, Crit Rev Ther Drug Carrier Syst, 12:1-55, 1996; and EP 0 362 279 B1. Quillaia saponin has also been disclosed as an adjuvant by Scott et al., Int. Archs. Allergy Appl. Immun., 77: 409, 1985. QuilA and cholesterol containing liposomes are described in Lipford et al., Vaccine, 12: 73-80, 1994. Quil A immunogenic compositions are also described in Bomford, Int. Archs. Allergy appl. Immun., 63: 170-177, 1980; Bomford, Int. Archs. Allergy appl. Immun., 67: 127-131, 1982; Scott et al., Int. Archs. Allergy appl Immun., 77: 409-412, 1985.

It has long been known that enterobacterial lipopolysaccharide (LPS) is a potent stimulator of the immune system, although its use in adjuvants has been curtailed by its toxic effects. A non-toxic derivative of LPS, monophosphoryl lipid A (MPL), produced by removal of the core carbohydrate group and the phosphate from the reducing-end glucosamine, has been described by Ribi et al (1986, Immunology and Immunopharmacology of bacterial endotoxins, Plenum Publ. Corp., NY, p407-419).

A further detoxified version of MPL results from the removal of the acyl chain from the 3-position of the disaccharide backbone, and is called 3-O-Deacylated monophosphoryl lipid A (3D-MPL). It can be purified and prepared by the methods taught in GB 2122204B, which reference also discloses the preparation of diphosphoryl lipid A, and 3-O-deacylated variants thereof. A preferred form of 3D-MPL is in the form of an emulsion having a small particle size less than 0.2 μm in diameter, and its method of manufacture is disclosed in WO 94/21292. Aqueous formulations comprising monophosphoryl lipid A and a surfactant have been described in WO 98/43670A2.

The bacterial lipopolysaccharide derived adjuvants to be formulated in the immunogenic compositions of the present invention may be purified and processed from bacterial sources, or alternatively they may be synthetic. For example, purified monophosphoryl lipid A is described in Ribi et al 1986 (supra), and 3-O-Deacylated monophosphoryl or diphosphoryl lipid A derived from Salmonella sp. is described in GB 2220211 and U.S. Pat. No. 4,912,094. Other purified and synthetic lipopolysaccharides have been described (U.S. Pat. No. 6,005,099 and EP 0 729 473 B1; Hilgers et al., Int. Arch. Allergy. Immunol., 79:392-6, 1986; Hilgers et al., Immunology, 60:141-6,1987; and EP 0 549 074 B1). Particularly preferred bacterial lipopolysaccharide adjuvants are 3D-MPL and the β(1-6) glucosamine disaccharides described in U.S. Pat. No. 6,005,099 and EP 0 729 473 B 1.

Accordingly, the LPS derivatives that may be used in the present invention are those immunostimulants that are similar in structure to that of LPS or MPL or 3D-MPL. In another aspect of the present invention the LPS derivatives may be an acylated monosaccharide, which is a sub-portion to the above structure of MPL.

In certain embodiments, the immunogenic composition of the present invention may comprise an immunostimulatory oligonucleotide. An “immunostimulatory oligonucleotide” refers to an oligonucleotide that contains a cytosine/guanine dinucleotide sequence and potentiates immune responses. An immunostimulatory oligonucleotide of interest may be between 2 to 100 base pairs in size and typically contain a consensus mitogenic CpG motif represented by the formula: 5′ X₁ X₂ CGX₃ X₄ 3′, where C and G are unmethylated, X₁, X₂, X₃ and X₄ are nucleotides and a GCG trinucleotide sequence is not present at or near the 5′ and 3′ termini (see U.S. Pat. No. 6,008,200, Krieg et al., issued Dec. 28, 1999, herein incorporated by reference). Preferably, the immunostimulatory sequences range between 8 to 40 base pairs in size. The dose and protocol for delivery will vary with the specific agent that is selected.

In certain embodiments, the immunogenic composition of the present invention may comprise immune response stimulating glycopeptides that are a group of compounds related to and derived from N-acetylmuramyl-L-alanyl-D-isoglutamine, which was determined by Ellouz et al., Biochem. & Biophys. Res. Comm., 59: 1317, 1974 to be the smallest effective unit possessing immunological adjuvant activity in M. tuberculosis, the mycobacterial component of Freund's complete adjuvant. A number of dipeptide- and polypeptide-substituted muramic acid derivatives were subsequently developed and found to have immunostimulating activity. The immune response stimulating glycopeptides which may be used in the practice of this invention are disclosed in U.S. Pat. Nos. 4,094,971; 4,101,536; 4,153,684; 4,235,771; 4,323,559; 4,327,085; 4,185;089; 4,082,736; 4,369,178, 4,314,998 and 4,082,735; and 4,186,194. The glycopeptides disclosed in these patents are incorporated herein by reference and made a part hereof as if set out in full herein. The compounds of Japanese patent application Nos. J5 4079-227, J5 4079-228, and J5 41206-696 would also be useful in the practice of this invention.

Specific compounds that may be useful in the context of the present invention, include, but are not limited to: N-acetylmuramyl-L-alpha-aminobutyryl-D-isoglutamine; 6-O-stearoyl-N-acetylmuramyl-L-alpha-aminobutyryl-D-isoglutamine-N-acetylmuramyl-L-threonyl-D-isoglutamine; N-acetylmuramyl-L-valyl-D-isoglutamine; N-acetylmuramyl-L-alanyl-D-glutamine n-butyl ester; N-acetyl-desmethyl-D-muramyl-L-alanyl-D-isoglutamine; N-acetylmuramyl-L-alanyl-D-glutamine; N-acetylmuramyl-L-seryl-D-isoglutamine; N-acetyl(butylmuramyl)-L-.alpha.-aminobutyl-D-isoglutamine; and N-acetyl(butylmuramyl)-L-alanyl-D-isoglutamine.

An effective amount of immunostimulating glycopeptide is that amount which effects an increase in titer level when administered in conjunction with an antigen over that titer level observed when the glycopeptide has not been co-administered. As can be appreciated, each glycopeptide may have an effective dose range that may differ from the other glycopeptides. Therefore, a single dose range cannot be prescribed which will have a precise fit for each possible glycopeptide within the scope of this invention. However, as a general rule, the glycopeptide will preferably be present in the immunogenic composition in an amount of between 0.001 and 5% (w/v). A more preferred amount is 0.01 to 3% (w/v).

Formulation and Administration

An immunogenic composition according to the present invention may further comprise components or substances that are useful in formulating the composition. The substances suitable for the present invention include but are not limited to physiologically acceptable excipients, diluents, and additive agents such as an acidic salt, a basic salt, a neutral salt, a carbohydrate, a starch, a polyelectrolyte, biocompatible hydrophilic materials, swellable materials, a gelatin, an amine, a surfactant, an inorganic acid or base, an organic acid or base, an amino acid, a monomer, an oligomer, a polymer or a mixture thereof.

In certain embodiments, the substance may include, but is not limited to, sodium chloride, sodium phosphate, bile salts, ammonium sulfate, ammonium chloride, sodium carbonate or potassium carbonate, polyethylene glycol, polyoxoethylene alkyl ethers, trehalose, mannitol, sorbitol, dextrose, dextrin, sucrose, lactose, saccharides, polysaccharides, oligosaccharides, saccharin, carboxymethyl cellulose, hydroxypropyl cellulose, hydroxypropyl methyl cellulose, methyl cellulose or sodium starch glycolate, citric acid, lactic acid, glycolic acid, acetic acid, ascorbic acid, tartaric acid, malic acid, maleic acid, benzoic acid, arginine, glycine, threonine, choline, ethanolamine, protamine, sodium alginate, heparin, docusate sodium, glycerin, glycofurol, propylene glycol, polysorbate, povidone, or albumin.

Another component optionally for use in the composition of the present invention is a metabolizable, non-toxic oil, preferably one of 6 to 30 carbon atoms, including, but not limited to, alkanes, alkenes, alkynes, and their corresponding acids and alcohols, the ethers and esters thereof, and mixtures thereof. The oil may be any vegetable oil, fish oil, animal oil, or synthetically prepared oil which can be metabolized by the body of the subject to which the adjuvant will be administered and which is not toxic to the subject.

The optional oil component of this invention may be any long chain alkane, alkene, or alkyne, or an acid or alcohol derivative thereof, either as the free acid, its salt or an ester such as a mono-, di- or triester, such as the triglycerides and esters of 1,2-propanediol or similar poly-hydroxy alcohols. Alcohols may be acylated employing a mono- or poly-functional acid, for example, acetic acid, propanoic acid, citric acid or the like. Ethers derived from long chain alcohols, which are oils and meet the other criteria set forth herein may also be used.

The individual alkane, alkene, or alkyne moiety and its acid or alcohol derivatives will have 6-30 carbon atoms. The moiety may have a straight or branched chain structure. It may be fully saturated or have one or more double or triple bonds. Where mono or polyester- or ether-based oils are employed, the limitation of 6-30 carbons applies to the individual fatty acid or fatty alcohol moieties, not the total carbon count.

Sources for vegetable oils include nuts, seeds, and grains. Peanut oil, soybean oil, coconut oil, and olive oil, the most commonly available, exemplify the nut oils. Seed oils include safflower oil, cottonseed oil, sunflower seed oil, sesame seed oil, and the like. In the grain group, corn oil is the most readily available, but the oil of other cereal grains such as wheat, oats, rye, rice, triticale and the like may also be used.

The technology for obtaining vegetable oils is well developed and well known. The compositions of these and other similar oils may be found in, for example, the Merck Index, and source materials on foods, nutrition and food technology.

The 6-10 carbon fatty acid esters of glycerol and 1,2-propanediol, while not occurring naturally in seed oils, may be prepared by hydrolysis, separation and esterification of the appropriate materials starting from the nut and seed oils. These products are commercially available under the name NEOBEE® from PVO International, Inc., Chemical Specialities Division, 416 Division Street, Boongon, N.J. and others. U.S. Pat. No. 4,772,466 is incorporated herein.

Oils from any animal source, including birds, may be employed in the adjuvants and compositions of this invention. Animal oils and fats are usually solids at physiological temperatures due to the fact that they exist as triglycerides and have a higher degree of saturation than oils from fish or vegetables. However, fatty acids are obtainable from animal fats by partial or complete triglyceride saponification which provides the free fatty acids. Fats and oils from mammalian milk are metabolizable and may therefore be used in the practice of this invention. The procedures for separation, purification, saponification and other means necessary for obtaining pure oils from animal sources are well known in the art.

The oil component of these adjuvants and composition formulations will be present in an amount from 1% to 30% by weight but preferably in an amount of 1% w/w. It is most preferred to use a 5% w/w concentration of oil.

Any physiologically acceptable buffer may be used herein, but phosphate buffers are preferred. Other acceptable buffers such as acetate, tris, bicarbonate, carbonate, or the like may be used as substitutes for phosphate buffers.

The pH of the aqueous component will preferably be between 6.0-8.0 though it is preferable to adjust the pH of the system to 6.8 where that pH does not significantly reduce the stability of other composition components and is not otherwise physiologically unsuitable.

In certain embodiments, the aqueous portion of the immunogenic compositions is buffered saline. When these compositions are intended for parenteral administration, it is preferable to make up these solutions so that the tonicity, i.e., osmolality is essentially the same as normal physiological fluids in order to prevent post-administration swelling or rapid absorption of the composition because of differential ion concentrations between the composition and physiological fluids. It is also preferable to buffer the saline in order to maintain a pH compatible with normal physiological conditions. Also, in certain instances, it may be necessary to-maintain the pH at a particular level in order to insure the stability of certain composition components such as the glycopeptides.

The quantity of buffered saline employed in these compositions will be that amount necessary to bring the value of the composition to unity. That is, a quantity of buffered saline sufficient to make 100% will be mixed with the other components listed above in order to bring the composition to volume.

In certain embodiments, the immunogenic composition of the present invention may comprise a surfactant. The term “surfactant” refers to non-toxic surface active agents capable of stabilizing the emulsion. There are a substantial number of emulsifying and suspending agents generally used in the pharmaceutical sciences. These include naturally derived materials such as gums, vegetable protein, alginates, cellulose derivatives, phospholipids (whether natural or synthetic), and the like. Certain polymers having a hydrophilic substituent on the polymer backbone have surfactant activity, for example, povidone, polyvinyl alcohol, and glycol ether-based compounds. Compounds derived from long chain fatty acids are a third substantial group of emulsifying and suspending agents usable in this invention. Though any of the foregoing surfactants can be used so long as they are non-toxic, glycol ether-based surfactants are preferred. Preferred surfactants are non-ionic. These include polyethylene glycols (especially PEG 200, 300, 400, 600 and 900), Span®, Arlacel®, Tween®, Myrj®, Brij® (all available from ICI America Inc., Wilmington, Del.), polyoxyethylene, polyol fatty acid esters, polyoxyethylene ether, polyoxypropylene fatty ethers, bee's wax derivatives containing polyoxyethylene, polyoxyethylene lanolin derivatives, polyoxyethylene fatty glycerides, glycerol fatty acid esters or other polyoxyethylene acid alcohol or ether derivatives of long-chain fatty acids of 12-21 carbon atoms. The presently preferred surfactant is Tween® 80 (otherwise known as polysorbate 80 or polyoxyethylene 20 sorbitan monooleate), although it should be understood that any of the above-mentioned surfactants would be suitable after lack of toxicity is demonstrated.

The preparation of the single and blend component systems can involve the addition of a surfactant to the processing media and/or to a solution of the polymeric composition with the antigenic compound. The residue of such a surfactant will typically remain in the polymeric composition upon formation of an encapsulated agent. The surfactant can be cationic, anionic or nonionic. Examples of useful surfactants include but are not limited to carboxymethyl cellulose, gelatin, poly(vinyl pyrrolidone), poly(ethylene glycol), Tween 80, Tween 20, polyvinyl alcohol or mixtures thereof. The surfactant, preferably, should not hinder the biodegradation of the polymeric composition and release of the antigenic compound.

The immunogenic composition may be prepared as injectables, as liquid solutions or emulsions. The antigenic compound such as peptides and haptens and the carrier such as poly(amino acids) may be mixed with physiologically acceptable excipients which are compatible with the antigenic compounds and carriers. Examples of such excipients include water, saline, Ringer's solution, dextrose solution, Hank's solution, and other aqueous physiologically balanced salt solutions. Nonaqueous vehicles, such as fixed oils, sesame oil, ethyl oleate, or triglycerides may also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer, and Tris buffer, while examples of preservatives include thimerosal, o-cresol, formalin, and benzyl alcohol. Standard formulations can either be liquids or solids that can be taken up in a suitable liquid as a suspension or solution for administration to an animal. Thus, in a non-liquid formulation, the excipient can comprise dextrose, human serum albumin, preservatives. etc., to which sterile water or saline can be added prior to administration.

The carriers such as poly(amino acids) can be covalently conjugated with the antigenic compounds to create a water-soluble conjugate in accordance with methods well-known to those skilled in the art, usually by covalent linkage between an amino or carboxyl group on the antigenic compound and on the carriers such as poly(amino acids).

In an alternative preferred embodiment, the carriers such as poly(amino acids) in the immunogenic composition may be cross-linked with a multivalent ion, preferably using an aqueous solution containing multivalent ions of the opposite charge to those of the charged side groups of the polyphosphazene, such as multivalent cations if the poly(amino acids) have acidic side groups or multivalent anions if the poly(amino acids) have basic side groups. Preferably, the polymers are cross-linked by di and trivalent metal ions such as calcium, copper, aluminum, magnesium, strontium, barium, tin, zinc, and iron, organic cations such as poly(amino acid), or other polymers such as poly(ethyleneimine), poly(vinylamine) and polysaccharides.

The preparation of an immunogenic composition formulations is generally described in New Trends and Developments in Vaccines, edited by Voller et al., University Park Press, Baltimore, Md., 1978. Encapsulation within liposomes is described, for example, by Fullerton, U.S. Pat. No. 4,235,877. Conjugation of proteins to macromolecules is disclosed, for example, by Likhite, U.S. Pat. No. 4,372,945 and by Armor et al., U.S. Pat. No. 4,474,757.

The amount of the antigenic compound in each immunogenic composition dose is selected as an amount that induces an immune response without significant, adverse side effects in typical recipients of the immunogenic compositions. Such amount will vary depending upon which specific immunogen is employed and how it is presented. Generally, it is expected that each dose will comprise 1-1000 μg of protein, preferably 2-100 μg, most preferably 4-40 μg. An optimal amount for a particular vaccine can be ascertained by standard studies involving observation of appropriate immune responses in subjects. Following an initial administration, subjects may receive one or several booster immunizations adequately spaced.

The compositions of the present invention may be used for both prophylatic and therapeutic purposes. Accordingly, in a further aspect, the invention therefore provides use of an immunogenic composition of the invention for the treatment of human patients, for use in veterinary settings, or for antibody production for diagnostic and therapeutic uses. The invention provides a method of treatment comprising administering an effective amount of an immunogenic composition of the present invention to a patient. In particular, the invention provides a method of treating viral, bacterial, parasitic infections or cancer which comprises administering an effective amount of an immunogenic composition of the present invention to a patient.

For oral preparations, the antigenic compound and carrier such as poly(amino acids) can be used alone or in combination with appropriate additives to make tablets, powders, granules or capsules, for example, with conventional additives, such as lactose, mannitol, corn starch or potato starch; with binders, such as crystalline cellulose, cellulose derivatives, acacial, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

In certain embodiments, the antigenic compound and carrier such as poly(amino acids) can be formulated into preparations for injections by dissolving, suspending or emulsifying them in an aqueous or nonaqueous solvent, such as vegetable or other similar oils, synthetic aliphatic acid glycerides, esters or higher aliphatic acids or propylene glycol; and if desired, with conventional additives such as solubilizers, isotonic agents, suspending agents, emulsifying agents, stabilizers and preservatives.

In certain embodiments, the antigenic compound and carrier such as poly(amino acids) of the present invention can be utilized in aerosol formulation to be administered via inhalation. The compounds of the present invention can be formulated into pressurized acceptable propellants such as dichlorodifluoromethane, propane, nitrogen and the like.

In certain embodiments, the antigenic compound and carrier such as poly(amino acids) may be formulated into an implant. Implants for sustained release formulations are well-known in the art. Implants are formulated as microspheres, slabs, etc. with biodegradable or non-biodegradable polymers. For example, polymers of lactic acid and/or glycolic acid form an erodible polymer that is well-tolerated by the host. The implant is placed in proximity to the site of response, where applicable, so that the local concentration of active agent is increased relative to the rest of the body.

The term “unit dosage form”, as used herein, refers to physically discrete units suitable as unitary dosages for human and animal subjects, each unit containing a predetermined quantity of compounds of the present invention calculated in an amount sufficient to produce the desired effect in association with a pharmaceutically/physiologically acceptable diluent, carrier or vehicle. The specifications for the novel unit dosage forms of the present invention depend on the particular compound employed and the effect to be achieved, and the pharmacodynamics associated with each compound in the host. Unit dosage forms for injection or intravenous administration may comprise the compound of the present invention in a composition as a soluble in sterile water, normal saline or another pharmaceutically acceptable carrier.

Physiologically acceptable excipients, such as vehicles, adjuvants, carriers or diluents, are readily available to the public. Moreover, physiologically acceptable auxiliary substances, such as pH adjusting and buffering agents, tonicity adjusting agents, stabilizers, wetting agents and the like, are readily available to the public

In certain embodiments, the immunogenic compositions may be a controlled release formulation comprising biodegradable polymer microspheres wherein an immunogenic composition is suspended in a polymer matrix, said polymer matrix being formed from at least two highly water soluble biodegradable polymers, and said microspheres being coated with a (d,1 lactide-glycolide) copolymer.

In one embodiment, the polymers are selected from the group consisting of starch, crosslinked starch, ficoll, polysucrose, polyvinyl alcohol, gelatine, hydroxymethyl cellulose, hydroxyethyl cellulose, hydroxypropyl cellulose, hydroxypropyl-ethyl cellulose, hydroxypropyl-methyl cellulose, sodium carboxymethyl cellulose, cellulose acetate, sodium alginate, polymaleic anhydride esters, polyortho esters, polyethyleneimine, polyethylene glycol, methoxypolyethylene glycol, ethoxypolyethylene glycol, polyethylene oxide, poly(1,3 bis(p-carboxyphenoxy) propane-co-sebacic anhydride, N,N-diethylaminoacetate, block copolymers of polyoxyethylene and polyoxypropylene.

An example of a suitable polyortho ester is 3,9-bis(methylene)-2,4,8,10,-tetra oxaspiro[5,5]undecane/1,6 hexanediol poly (ortho ester).

It is preferred that the weight ratio of the two polymers is in the range of from 20:80 to 80:20.

In another embodiment, the polymer matrix is selected from starch and ficoll, starch and polysucrose, starch and polyvinyl alcohol, starch and gelatine, hydroxyethyl cellulose and hydroxypropyl cellulose, gelatine and hydroxyethyl cellulose, gelatine and polyvinyl alcohol, polysucrose and polyvinyl alcohol, and sodium carboxymethyl cellulose and sodium alginate.

When the polymer matrix comprises starch and ficoll, the preferred weight ratio of starch to ficoll is preferably from 85:15 to 60:40, and more preferably from 75:25 to 65:35.

Partially synthetic cellulose esters, polyvinylpyrrolidone and poly-6-aminohexanoic acid as well polyvinylalcohol, alkali and ammonium alginate, methylcellulose, ethylcellulose, hydroxyethylcellulose, ethylhydroxyethylcellulose, and sodium-carboxymethylcellulose have particularly proven their value as water-soluble polymers, which are to be used pursuant to the invention.

In the compositions of this invention, the component(s) of the polymeric composition are preferably biocompatible, which term is known in the art to include that the components are substantially non-toxic, non carcinogenic, and should not substantially induce inflammation in body tissues upon administration.

The biodegradable polymer is used in an amount ranging from 1 to 100, preferably, from 5 to 30 times the weight of the core particle. The coating of the core particle is made of a water-soluble substance which is insoluble in the organic solvent, and therefore, it prevents the reduction or loss of the antigenicity of the antigen by blocking the contact of the antigen with the organic solvent.

Exemplary hydrophobic biodegradable polymers which may be used in the present invention include poly(lactide-co-glycolide).(PLGA), polyglycolide(PGA), polylactide(PLA), copolyoxalates, polycaprolactone, poly(lactide-co-caprolactone), polyesteramides, polyorthoesters, poly(p-hydroxybutyric acid), and polyanhydride; while PLGA and PLA are preferred.

Any of the organic solvents well-known in the art may be used to dissolve the biodegradable polymer, and these include carbon tetrachloride, methylene chloride, acetone, chloroform, ethyl acetate and acetonitrile.

General techniques for the preparation of the antigenic compound and carrier encapsulated structures are known to those of skill in the art. See, e.g., U.S. Pat. No. 5,407,609 to Tice et al., Grandfils et al. (Journal of Controlled Release, 1996, Peyer's patch 109-122), Bodmeier et al.(International Journal of Pharmaceutics, 51: 1-8,1989), and European Patent No. A1 0,058, 481 to Hutchinson.

In certain embodiments, the antigenic compound and carrier such as poly(amino acids) of the present invention can be formed into a core particle that may be coated by a biodegradable polymer. U.S. Pat. No. 5,753,234 is incorporated herein by reference. The core particle is prepared by dissolving or dispersing the antigenic compound and carrier such as poly(amino acids) in a solution obtained by dissolving a water-soluble substance in a suitable aqueous solvent, e.g., water or a buffer, and drying the mixture by a spray drying or a freeze drying method. A additionally suitable adjuvant or adjuvants may be added to the solution, if necessary, and examples thereof include alum; muramyl dipeptide, muramyl tripeptide and derivatives thereof; tymosin alpha; monophosphoryl lipid A; saponin; an immunostimulating complex; a polyelectrolyte such as a copolymer of polyoxyethylene and polyoxypropylene; and a mixture thereof.

The water-soluble substance used for the preparation of the core particle does not bring about an undesirable interaction with the antigenic compound and carrier such as poly(amino acids) and is practically insoluble in the organic solvent used in the coating step. Any water-soluble substance is contemplated so long that it does not bring into the vaccine composition any undesirable effects, including antigenicity and local toxicity. Exemplary water-soluble substances include water-soluble saccharides such as glucose, xylose, galactose, fructose, lactose, maltose, saccharose, alginate, dextran, hyaluronic acid, chondroitin sulfate and water-soluble cellulose derivatives, e.g., hydroxypropylmethyl cellulose, hydroxypropyl cellulose (HPC), carboxymethyl cellulose (CMC) and sodium carboxymethyl cellulose (CMC-Na); amino acids such as glycine, alanine, glutamic acid, arginine, lysine and a salt thereof; and a mixture thereof; while HPC, CMC, CMC-Na, gelatin, and a mixture thereof are preferred.

The water-soluble substance may be used in an amount ranging from 1 to 50, preferably, from 5 to 15 times the weight of total antigen.

The core particle so prepared has a particle size ranging from 0.1 to 200 μm, preferably, from 0.5 to 30 μm. In order to prepare the final microparticle, the core particle is dispersed in an organic solvent, wherein a hydrophobic biodegradable polymer is dissolved, by using a suitable apparatus, e.g., a magnetic stirrer, homogenizer, microfluidizer and sonicator.

Specifically, a microparticle of the present invention may be prepared from the core particle dispersed system in accordance with any one of the following conventional methods.

1) Solvent Evaporation Method

This method is well known for the preparation of a microparticle, but the present invention differs from the prior arts in that the core particle dispersed system, wherein the contact of the antigen with the organic solvent is prevented, is employed in place of an aqueous solution wherein the antigen is dissolved or dispersed.

Specifically, the microparticle may be prepared by dispersing the core particle dispersed system in an aqueous solution comprising a surfactant to obtain an O/W emulsion and then removing the organic solvent from the core particle dispersed system, or by dispersing the core particle dispersed system in a solvent, which is immiscible with the core particle dispersed system and is a nonsolvent for the biodegradable polymer, to prepare an 0/0 emulsion and removing the organic solvent from the core particle dispersed system. When acetonitrile is used as the organic solvent of the core particle dispersed system, a mineral oil can be used as the solvent which is immiscible with the core particle dispersed system and is a nonsolvent for the biodegradable polymer

2) Solvent Extraction Method

This method is also well-known in the art for the preparation of a microparticle, but the present invention differs from the prior arts in that the core particle dispersed system is employed. Specifically, the microparticle may be prepared by extracting the organic solvent of the core particle dispersed system by using a solvent, which is immiscible with the core particle dispersed system and is a nonsolvent for the biodegradable polymer, such as mineral oil or paraffin oil.

3) Rapid Freezing and Solvent Extraction Method

The present invention is different from the prior arts in that the core particle dispersed system is employed. Specifically, the core particle dispersed system is sprayed into a low-temperature liquid gas phase using an ultrasonic apparatus to form a frozen particle. This particle is collected on the surface of frozen ethanol. As the frozen ethanol is melted, the frozen particle thaws and the organic solvent in the particle is extracted into the ethanol phase with concomitant formation of a microparticle coated with the biodegradable polymer.

4) Spray Drying Method

This method is most preferable for use in the present invention and, specifically, the microparticle is prepared by spraying the core particle dispersed system by employing a spray-dryer. This method is advantageous due to its high productivity and rapidity. Further, it is also advantageous in that removal of water is unnecessary because water is not used in the process; no surfactant is required; and the washing and drying processes can be omitted.

The particle size of the microparticle thus prepared ranges from 0.5 to 300 μm, preferably, from 1 to 180 μm. Those microparticles having a particle size smaller than 180 μm may be dispersed in an injection medium to prepare an injection formulation for subcutaneous, intramuscular, and intraperitoneal injections. Those particles having a particle size larger than 180 μm may be used for preparing a formulation for oral administration.

Therefore, the present invention further provides a single-shot immunogenic formulation that is prepared by dispersing the microparticles in a suitable injection medium. The immunogenic formulation may comprise single antigenic compound alone, or two or more kinds of antigenic compounds together. The immunogenic formulation comprising two or more antigenic compounds may be prepared by employing core particles comprising a mixture of two or more kinds of antigenic compounds, or by employing a mixture of two or more kinds of core particles each comprising an antigenic compound different from each other.

In certain embodiments, the immunogenic compositions of the present invention comprise biodegradable polymers such that the antigenic compound and carrier such as poly(amino acids) are suspended in the biodegradable polymers. The suspension can be manufactured into a controlled release microspheres or microparticles. Exemplary poly (lactide-glycolide) can be used for the present invention. The selection of the particular (d,1 lactide-glycolide) copolymer will depend in a large part on how long a period the microsphere is intended to release the active ingredient. For example, a (d,1. lactide-glycolide) copolymer made from about 80% lactic acid and 20% glycolic acid is very stable and would provide a microsphere suitable for release of active ingredient over a period of weeks. A (d,1 lactide-glycolide) copolymer made from 50% lactic acid and 50% glycolic acid is stable and would provide an microsphere suitable for release of active ingredient over a period of days. A (d,1 lactide-glycolide) copolymer made from 20% lactic acid and 80% glycolic acid disintegrates relatively easily and would provide an microsphere suitable for release of active ingredient over a period of 1-2 days. The coating makes the microspheres more resistant to enzymatic degradation.

In any composition of this invention used for inducing or potentiating an immune response, the immune response that is induced or potentiated is a CTL, T helper cell or neutralizing antibody response. In some embodiments, the antigen can be a nucleic acid functionally encoding such an antigen. A nucleic acid functionally encoding an antigen is a nucleic acid capable of expression of the antigen in the cells into which the nucleic acid will be taken up; for example, such a nucleic acid molecule will have appropriate expression controls (e.g., promoter, enhancer, if desired, translation start codon, polyadenylation signal etc.) and codon usage compatible with the cells.

The immunogenic composition of the present invention can be administered to a subject using a variety of methods known in the art. In one embodiment, the immunogenic composition can be delivered parenterally, by injection, such as intramuscular, intraperitoneal, intravenous or subcutaneous injection, or by inhalation. In another embodiment, the immunogenic composition can be delivered rectally, vaginally, nasally, orally, opthamalically, topically, transdermally or intradermally. When the mode of administration is by injection, the encapsulated antigenic compound may stay at the injection site for up to two weeks, thus providing a depot of antigen that will give sustained release or pulsatile release in vivo. Such a delivery system may allow single-shot immunogenic formulations to be produced for antigenic compounds which would otherwise require multiple injections to elicit an immune response.

For parenteral administration in an aqueous solution, for example, the solution should be suitably buffered if necessary and the liquid diluent first rendered isotonic with sufficient saline or glucose. These particular aqueous solutions are especially suitable for intravenous and intraperitoneal administration. In this connection, sterile aqueous media which can be employed will be known to those of skill in the art in light of the present disclosure. Exemplary injection media which can be used in the present invention include a buffer with or without dispersing agents and/or preservatives, an edible oil, mineral oil, cod liver oil, squalene, squalane, mono-, di- or triglyceride and a mixture thereof; said edible oil being corn oil, sesame oil, olive oil, soybean oil, safflower oil, cotton seed oil, peanut oil or a mixture thereof.

The exact amount of such compositions required will vary from subject to subject, depending on the species, age, weight and general condition of the subject, the severity of the disease, infection or condition that is being treated or prevented, the particular compound used, its mode of administration, and the like. Thus, it is not possible to specify an exact amount. However, an appropriate amount may be determined by one of ordinary skill in the art using only routine experimentation given the teachings herein. In one embodiment, the amount of antigenic compound that is administered in an encapsulated form is from 1 ng to 5 mg. In another embodiment, the amount of antigenic compound that is administered in an encapsulated form is from 1 mg to 100 mg. In another embodiment, the amount of antigenic compound that is administered in an encapsulated form is at least about 10 mg. In another embodiment, the amount of antigenic compound that is administered in an encapsulated form is from 1 ng to 10 mg. A single administration may be sufficient, depending upon the disease, condition, or infection being treated or prevented; however, it is also contemplated that multiple administrations may be administered. Administrations after the initial administration may be of lower dosage than the initial dosage.

Methods of Use

The compositions of the invention may be used for a variety of purposes, including uses related to the production of an immune response, particularly a sustained immune response, against an antigen or hapten. Further, once weakly antigenic or non-antigenic tumor, viral, or bacterial antigens are made more antigenic by the carrier of the present invention, such carrier-antigenic compound complexes can be delivered to a patient to elicit an immune response directed to the heretofore weakly or non-immunogenic antigens thereby ameliorating or treating the disease associated therewith.

In one embodiment, a composition of the invention is used to generate an immune response directed against a substantially non-immunogenic antigen or hapten. Accordingly, the invention provides a method for inducing or enhancing an immune response by administering to an animal, such as, for example, a mammal, an effective amount of a composition of the invention. In one embodiment, the administered composition is an antigen/hapten conjugated to a carrier of the invention. In another embodiment, a composition of the invention comprises an antigen/hapten and a non-conjugated carrier used as an adjuvant. The presence of the carrier facilitates the generation or enhancement of an immune response, such as antibodies or T-cells, directed against a antigen or hapten. In certain embodiments, the resulting immune response will be primarily directed against or specific for the antigen/hapten, rather than the carrier.

In certain embodiments, a method of the invention for inducing or enhancing an immune response may be used to generate a protective immune response in an animal, such as a mammal. Thus, a composition of the invention may be used as a vaccine. The protective immune response may be either cell-mediated or humoral, or both. In one embodiment, the immune response is directed against an infectious agent and prevents or inhibits further infection, or lessens or ameliorates a symptom or biologic characteristic of a disease or condition associated with infection. In another embodiment, the immune response may be targeted against a tumor and may lessen or ameliorate certain symptoms or biological characteristics associated with the presence of the tumor.

In other embodiments, a composition of the invention may be used to treat a patient with a disease, infection, or pathologic condition. Accordingly, the invention provides methods of treating a disease or pathologic condition by administering to an animal, for example, a mammal, an effective amount of a composition of the invention. In certain embodiments, the method may be used to treat a microbial infections caused by a virus, yeast, fungus, or bacteria, for example.

In addition to directly treating a tumor or other pathological condition, methods of the invention may be used to elicit or enhance an immune response to thereby indirectly treat a tumor or pathological condition, for example, by directing a destructive immunological attack on a tumor by activating biological effector functions or blocking key molecules necessary for tumor growth, metastasis, or angiogenesis.

In addition, the compositions have general utility for producing antibodies to antigens/haptens for diagnostic and therapeutic uses as well as providing a means by which a therapeutic immune response can be generated in vivo in a vertebrate animal in need of treatment. In one example the antigen may be a tumor antigen. In such a case the presence or absence of a cancer in a patient may be determined by (a) contacting a biological sample obtained from a patient with an antibody produced by using the carrier of the present invention; (b) detecting in the sample a level of polypeptide that binds to the antibody; and (c) comparing the level of polypeptide with a predetermined cut-off value.

In a preferred embodiment, assays like those set forth above involve the use of the antibody immobilized on a solid support to bind to and remove the polypeptide from the remainder of the sample. The bound polypeptide may then be detected using a detection reagent that contains a reporter group and specifically binds to the antibody/polypeptide complex. Such detection reagents may comprise, for example, an antibody that specifically binds to the polypeptide or an antibody or other agent that specifically binds to the antibody, such as an anti-immunoglobulin, protein G, protein A or a lectin. Alternatively, a competitive assay may be utilized, in which a polypeptide is labeled with a reporter group and allowed to bind to the immobilized antibody after incubation of the antibody with the sample. The extent to which components of the sample inhibit the binding of the labeled polypeptide to the antibody is indicative of the reactivity of the sample with the immobilized antibody. Suitable polypeptides for use within such assays include full length colon tumor proteins and polypeptide portions thereof to which the antibody binds, as described above.

The solid support may be any material known to those of ordinary skill in the art to which the tumor protein may be attached. For example, the solid support may be a test well in a microtiter plate or a nitrocellulose or other suitable membrane. Alternatively, the support may be a bead or disc, such as glass, fiberglass, latex or a plastic material such as polystyrene or polyvinylchloride. The support may also be a magnetic particle or a fiber optic sensor, such as those disclosed, for example, in U.S. Pat. No. 5,359,681. The antibody may be immobilized on the solid support using a variety of techniques known to those of skill in the art, which are amply described in the patent and scientific literature. In the context of the present invention, the term “immobilization” refers to both noncovalent association, such as adsorption, and covalent attachment (which may be a direct linkage between the agent and functional groups on the support or may be a linkage by way of a cross-linking agent). Immobilization by adsorption to a well in a microtiter plate or to a membrane is preferred. In such cases, adsorption may be achieved by contacting the antibody, in a suitable buffer, with the solid support for a suitable amount of time. The contact time varies with temperature, but is typically between about 1 hour and about 1 day. In general, contacting a well of a plastic microtiter plate (such as polystyrene or polyvinylchloride) with an amount of antibody or antigen ranging from about 10 ng to about 10 μg, and preferably about 100 ng to about 1 μg, is sufficient to immobilize an adequate amount of antibody or antigen.

Of course, numerous other assay protocols exist that are suitable for use with the antibodies produced by the present invention. The above descriptions are intended to be exemplary only. For example, it will be apparent to those of ordinary skill in the art that the above protocols may be readily modified to use viral or bacterial polypeptides or haptens to detect for their presence in a biological sample.

Certain in vivo diagnostic assays may be performed directly to detect bacteria, virus, tumors, or virtually anything from which the weakly or non-antigenic agent is derived from. One such assay involves contacting tumor cells with an antibody produced by utilizing the carrier compositions of the present invention. The bound antibody may then be detected directly or indirectly via a reporter group. Such antibodies may also be used in histological applications.

As the herein described compositions allow for the production of antibodies to molecules that previously escaped antibody recognition or that were only weakly antigenic, an entire new set of antigens may gain use by utilizing the present invention. For example, any cell surface markers that meet this criteria can now be used in combination with the present invention to develop highly specific antibodies thereto, which besides therapeutic applications also provides the ability to utilize such antibodies for cell sorting methodologies.

The present invention further provides kits for use within any of the above diagnostic, therapeutic methods, or methods of producing antibodies or an immune response. Such kits typically comprise two or more components necessary for performing a diagnostic assay. Components may be instructions for use, compounds, reagents, containers and/or equipment. For example, one container within a kit may contain a substantially non-antigenic polymer and a hapten or weakly or non-antigen agent. One or more additional containers may enclose elements, such as reagents or buffers, to be used in the assay or method. Such kits may also, or alternatively, contain a detection reagent as described above that contains a reporter group suitable for direct or indirect detection of antibody binding.

There are a variety of assay formats known to those of ordinary skill in the art for using an antibody to detect polypeptide markers in a sample. See, e.g., Harlow and Lane, Antibodies: A Laboratory Manual, Cold Spring Harbor Laboratory, 1988. However, some of these markers, be they viral envelope proteins, bacterial proteins, or tumor antigens are only weakly antigenic or non-antigenic. However, utilizing the compositions of the present invention antibodies to such markers can be made in animals and used for diagnostic agents.

The following Examples are offered by way of illustration and not by way of limitation.

### EXAMPLES

### Example 1

### PREPARATION OF POLY-L-GLUTAMIC ACID

Poly-L-glutamic acid sodium salt (85.9 g) (Sigma Chemical Co., 37 kD MW determined by viscosity measurement) was dissolved in USP putified water (534 ml; 6.2 ml/g), and the solution was cooled to between 0° C.-5° C. Dilute hydrochloric acid solution (1 M) was added dropwise with vigorous stirring keeping the temperature <10° C. until the pH was between pH 2 to 2.5. During the addition, the poly-L-glutamic acid separated out of solution. The reaction mixture was warmed to room temperature and stirred for 1 hour. The suspension was centrifuged at 2700×g for 10 minutes. He upper aqueous layer was removed and the solid was resuspended in 560 ml USP purified water and recentrifuged for 10 minutes. The upper aqueous layer was removed and the pH was measured. Washing was continued, if necessary, until the pH of the aqueous layer was ≧3.0. The wet solid was lyophilized on a LABCONCO™ freeze dry system until a constant weight was obtained. The wt % sodium was no greater than 7000 ppm as determined by ICP.

### Example 2

### GENERATION OF AN ANTIGEN SPECIFIC IMMUNE RESPONSE

This example demonstrates the immune response specifically generated against a Plasmodium falciparum peptide following immunization with this peptide conjugated to a polyglutamate carrier.

Materials and Methods

A peptide derived from Plasmodium falciparum (peptide EXP1 82; Doolan et al J. Immunol2000, 165:1123-1137) with the sequence NH₂-AGLLGNVSTVLLGGV-COOH was synthesized by Genemed Synthesis Inc. (San Grancisco, Calif.). Purity and sequence of the peptide (lot#10013321) was determined by HPLC, amino acid analysis and mass spectrometry. The peptide was then coupled to the gamma-carboxyl group of glutamyl residues within the polyglutamate homopolymer backbone of CT-2103, 37% (by weight) paclitaxel lot# 1116-91). The resulting CT-2103:peptide conjugate, called CP1 PaTXL, (lot# 1172-74) was then sent to Covance Research Products (Denver, Pa.) for hybridoma development.

The hybridoma project began with a series of sequential inoculations by subcutaneous (SC) or interperitoneal (IP) injections of 100 μg of CPT1PaTXL formulated in PBS. In addition to the CPT1PaTXL, the first two inoculations also contained Bacillus pertussis toxin as adjuvant (PTA). All subsequent inoculations were without PTA and had various amounts of CPT1PaTXL according to the following approximate schedule:

| Day | Procedure |
|---|---|
| 0 | Pre-bleed, innoculate SC/IP with 100 μg CPT1PaTXL (+PTA) |
| 21 | Boost SC/IP 100 μg CPT1PaTXL (+PTA) |
| 31 | Test Bleed |
| 32 | ELISA test (decision point to go on or terminate non-responders) |
| 42 | Boost SC/lP 50 μg CPT1PaTXL |
| 52 | Test Bleed |
| 53 | ELISA test |
| 63 | Boost SC/IP 50 μg CPT1PaTXL |
| 73 | Test Bleed |
| 74 | ELISA test |
| 84 | Boost SC/IP 50 μg CPT1PaTXL |
| 94 | Test Bleed |
| 95 | ELISA test |
| 98 | Boost IV 10 μg CPT1PaTXL (1 mouse only, this is a pre-fusion |
| | boost) |
| 99 | Boost IV 5 μg CPT1PaTXL (1 mouse only, this is a pre-fusion |
| | boost) |
| 100 | Boost IV 5 μg CPT1PaIXL (1 mouse only, this is a pre-fusion |
| | boost) |
| 101 | Terminal Bleed and begin fusion |

Ten mice were used in the immunization protocol. At each scheduled test bleed, 100 μl of blood was obtained from the tail, and an ELISA was conducted to evaluate reactivity against either Polyglutamate (PG) alone, Paclitaxel (TXL) or CT-2103. Because of the poor solubility of TXL in aqueous solutions, TXL was coupled to Bovine Serum Albumin, fraction V (Sigma Biochemicals, St. Louis Mo.) for ELISA testing. ELISA assays are performed as described in detail in Harlow, E. and Lane, D., Antibodies, A Laboratory Manual, Cold Spring Harbor Laboratory Press, 1988, pp. 564-569. Reactivity against the P. falciparum peptide epitope was analyzed following the terminal bleed.

Results

There was no detectable anti-PG or anti-TXL reactivity from any mouse at any bleed, including the terminal bleed. All of the mice showed increasing titers of anti-CT-2103 reactivity over the course of the immunization protocol. One mouse, CT63, was selected for hybridoma development due to its robust anti-CT-2103 response. ELISA tests were performed to determine the anti-CT-2103 titers of this mouse, as well as the titer to P. falciparum peptide in the terminal antisera. The reactivity of mouse CT63 antisera to the P. falciparum epitope was equivalent to that of CT-2103 and was detectable to dilutions in excess of 300000⁻¹ (data not shown). Reactivity against either PG or CT-2103 was also tested by Western blotting. As little as 2 ng of CT-2103 was detected, whereas PG was undetectable at any concentration tested (data not shown).

### EXAMPLE 3

### HUMORAL AND CELLULAR IMMUNE RESPONSES TO HAPTENS COUPLED TO POLYGLUTAMATE CARRIERS

This example illustrates experimental procedures to evaluate both humoral and cellular immune responses directed against a hapten fused to a carrier of the invention and the carrier itself.

Materials and Methods

To evaluate the immune response directed against the antigen, prostate specific antigen (PSA), and the carriers, polyglutamate (PG) and keyhole limpet hemocyanin (KLH), animals are inoculated with one of the following immunogens: (1) PSA; (2) PSA coupled to PG (PG-PSA); (3) PG alone; (4) PSA coupled to KLH (KLH-PSA); or KLH alone, in combination with Complete Freund's Adjuvant (CFA). The animals are then challenged up to four times with the same immunogen, in combination with Incomplete Freund's Advujant (IFA). Each boost (challenge) with the immunogen occurs 21 days after inoculation or each subsequent boost, according to the regiment presented below.

Treatment Regimen:

| | |
|---|---|
| Day 1 | Pre-bleed and inoculation with 50 to 100 μg of immunogen |
| Day 22 | Bleed and boost with 50 to 100 μg of immunogen |
| Day 43 | Bleed and boost with 50 to 100 μg of immunogen |
| Day 64 | Bleed and boost with 50 to 100 μg of immunogen |
| Day 85 | Bleed and boost with 50 to 100 μg of immunogen |

Animals are then exsanguinated, immune organs are removed, and T cells are isolated. Antisera re tested by ELISA for reactivity against each of the different immunogens described above. Isolated T cells are stimulated in a CTL assay. Irradiated splenic adherent cells (SadC) from untreated littermates are used as controls. SadC are used as antigen presenting cells for stimulating T cell responses from T cells derived from treated animals. Proliferative responses and lymphokine and cytokine levels from responding T cells are used as a measure of cellular response to each immunogen.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A composition for enhancing the immunogenicity of a hapten comprising a substantially non-antigenic carrier and the hapten, wherein the carrier is associated with the hapten.

2. The composition of claim 1, wherein said association of the hapten and the carrier is by charge-charge interaction.

3. The composition of claim 1, wherein said association of the hapten and the carrier is a covalent bond.

4. The composition of claim 3, wherein said covalent bond is a peptide bond.

5. The composition of claim 1, wherein said carrier is a polymer.

6. The composition of claim 5, wherein said polymer is a polyamino acid polymer.

7. The composition of claim 6, wherein said polyamino acid polymer is a polyanionic amino acid polymer.

8. The composition of claim 7, wherein said polyanionic amino acid polymer is a polyglutamate polymer.

9. The composition of claim 5, wherein said polymer is selected from the group consisting of polyanionic amino acids, polyglycolide, polylactide, poly(p-dioxanone), polycaprolactone, polyhydroxyalkanoates, poly(propylene fumarate), poly(ortho esters), polyanhydrides, polyphosphazenes, poly(alkylcyanoacrylates), poloxamers, polyglutamate, polyethylene glycol.

10. The composition of claim 5, wherein said polymer is biodegradable.

11. The composition of claim 5, wherein said polymer has a molecular weight of between ten and 5,000 KD.

12. The composition of claim 11, wherein said polymer has molecular weight of between 100 and 1,000 KD.

13. The composition of claim 12, wherein said polymer is about 600 KD.

14. The composition of claim 1 or 5, further comprising a physiologically acceptable excipient or diluent.

15. The composition of any one of claim 1 or 5, further comprising an adjuvant.

16. The composition of claim 15, wherein said adjuvant is selected from the group consisting of: liposomes, oily phases, Freund's adjuvant, inorganic salts, cytokines, chemokines, growth factors, angiogenic factors, apoptosis inhibitors, hormones, immunomodulators, plasmid DNA, poly inosine:cytosine, immunostimulatory oligonucleotides, bacterial agents, listeriolysin, streptolysin, mineral oil, non-mineral oil, self-emulsifiable oil, pertussis toxin mutants, saponins, lipopolysaccharide, monophosphoryl lipid A, and N-acetylmuramyl-L-alanyl-D-isoglutamine and related compounds.

17. The composition of any one of claims 1 or 5, wherein said hapten is selected from the group consisting of: (1) live, heat-killed, or chemically attenuated microbes; (2) fragments, extracts, subunits, metabolites, and recombinant constructs of microbes and mammalian proteins, glycoproteins and epitopes; (3) tumor antigens; and (4) nucleic acid molecules.

18. The composition of claim 17, wherein said tumor antigen is selected from the group consisting of: cancinoembryonic antigen, carcinoembryonic antigen peptide-1 (CAP-1), α-fetoprotein, alkaline phosphatase isoenzyme, prostate-specific antigen, beta subunit of choriogonadotropic hormone, calcitonin, Bence-Jones proteins, aspartyl β-hydroxylase, NY-ESO-1, 707 alanine proline, adenocarcinoma antigen (ART-4), B antigen (BAGE), β-catenin, m-catenin, Bcr-abl, CTL-recognized antigen on melanoma (CAMEL), caspase-8, CDC27, CDK4, cancer/testis antigen, cyclophilin B, differentiation antigens melanoma (DAM-6 and DAM-10), elongation factor 2, Ets, glycoprotein 250, G antigen, N-acetylglucosaminyltransferase V, glycoprotein 100 kD, helicose antigen, human epidermal receptor-2/neurological (HER2-neu), HLA-A2 R170I, human papilloma virus E7, heat shock protein 70—2 mutated, human signet ring tumor—2, human telomerase reverse transcriptase, intestinal carboxyl esterase, L antigen, low density lipid receptor/GDP-L-fucose:-D-galactosidase 2-L-fucosyltransferase, melanoma antigen, Melanoma antigen A, melanocortin 1 receptor, myosin mutated, mucin 1, melanoma ubiquitous mutated 1, 2, 3, NA cDNA clone of patient M88, New York—esophageous 1, protein 15, minor bcr-abl, promyelocytic leukaemia/retinoic acid receptor (Pml/RAR), preferentially expressed antigen of melanoma, prostate-specific antigen, prostate-specific membrane antigen, renal antigen, renal ubiquitous 1 or 2, sarcoma antigen, squamous antigen rejecting tumor 1 or 3, translocation Ets-family leukemia/acute myeloid leukemia 1 (TEL/AML1), triosephosphate isomerase mutated, gp75, tyrosinase related protein 2, TRP-2/intron, and Wilms' tumor gene.

19. A composition for enhancing the immunogenicity of an antigenic compound comprising a substantially non-antigenic carrier and the antigenic compound, wherein the carrier is associated with the antigenic compound.

20. The composition of claim 19, wherein said association of the compound and the carrier is by charge-charge interaction.

21. The composition of claim 19, wherein said association of the compound and the carrier is a covalent bond.

22. The composition of claim 21, wherein said covalent bond is a peptide bond.

23. The composition of claim 19, wherein said carrier is a polymer.

24. The composition of claim 23, wherein said polymer is a polyamino acid polymer.

25. The composition of claim 24, wherein said polyamino acid polymer is a polyanionic amino acid polymer.

26. The composition of claim 25, wherein said polyanionic amino acid polymer is a polyglutamate polymer.

27. The composition of claim 23, wherein said polymer is selected from the group consisting of polyanionic amino acids, polyglycolide, polylactide, poly(p-dioxanone), polycaprolactone, polyhydroxyalkanoates, poly(propylene fumarate), poly(ortho esters), polyanhydrides, polyphosphazenes, poly(alkylcyanoacrylates), poloxamers, polyglutamate, polyethylene glycol.

28. The composition of claim 23, wherein said polymer is biodegradable.

29. The composition of claim 23, wherein said polymer has a molecular weight of between ten and 5,000 KD.

30. The composition of claim 29, wherein said polymer has molecular weight of between 100 and 1,000 KD.

31. The composition of claim 30, wherein said polymer is about 600 KD.

32. The composition of any one of claims 19 or 23, further comprising a physiologically acceptable excipient or diluent.

33. The composition of any one of claims 19 or 23, further comprising an adjuvant.

34. The composition of claim 33, wherein said adjuvant is selected from the group consisting of: liposomes, oily phases, Freund's adjuvant, inorganic salts, cytokines, chemokines, growth factors, angiogenic factors, apoptosis inhibitors, hormones, immunomodulators, plasmid DNA, poly inosine:cytosine, immunostimulatory oligonucleotides, bacterial agents, listeriolysin, streptolysin, mineral oil, non-mineral oil, self-emulsifiable oil, pertussis toxin mutants, saponins, lipopolysaccharide, monophosphoryl lipid A, and N-acetylmuramyl-L-alanyl-D-isoglutamine and related compounds.

35. The composition of claim 19 or 23, wherein said antigenic compound is derived from an agent selected from the group consisting of a virus, a fungus, a bacteria, a diseased tissue, and a hapten.

36. The composition of claim 35, wherein said diseased tissue is a tumor.

37. The composition of claim 35, wherein said agent is a virus.

38. The composition of claim 19, wherein said antigenic compound is selected from the group consisting of peptides, proteins, polysaccharides, and haptens.

39. An immunogenic composition for enhancing the immunogenicity of an antigenic compound, comprising a substantially non-antigenic, biodegradable and soluble carrier, and the antigenic compound; wherein the antigenic compound is associated with the carrier.

40. The composition of claim 39, wherein the antigenic compound is covalently conjugated to the carrier.

41. The composition of claim 39, wherein the antigenic compound is associated with the carrier by charge interactions.

42. The composition of any of claims 1, 5, 19, or 39, further comprising a surfactant selecting from the group consisting of polyethylene glycols, including PEG 200, 300, 400, 600 and 900, Span®, Arlacel®, Tween®, including Tween® 80, Myrj®, Brij®, polyoxyethylene, polyol fatty acid esters, polyoxyethylene ether, polyoxypropylene fatty ethers, bee's wax derivatives containing polyoxyethylene, polyoxyethylene lanolin derivatives, polyoxyethylene fatty glycerides, glycerol fatty acid esters, polyoxyethylene acid alcohols, and ether derivatives of long-chain fatty acids of 12-21 carbon atoms.

43. An immunogenic composition for enhancing the immunogenicity of an antigenic compound, comprising a substantially non-antigenic polyanionic amino acid polymer, and an antigenic compound; wherein the antigenic compound is a peptide, polypeptide or a hapten, and wherein said peptide, polypeptide or a hapten is conjugated to the polyanionic amino acid polymer.

44. The composition of claim 43, wherein said polyanionic amino acid polymer is a homopolymer.

45. The composition of claim 44, wherein said homopolymer is a polyglutamate homopolymer.

46. The composition of claim 45, wherein said homopolymer has a molecular weight of between ten and 5,000 KD.

47. The composition of claim 46, wherein said polymer has molecular weight of between 100 and 1,000 KD.

48. The composition of claim 47, wherein said polymer is about 600 KD.

49. The composition of claim 43, wherein said conjugation is by a peptide bond.

50. A method for eliciting or enhancing an immune response to an antigenic compound, comprising administering to a mammal a substantially non-antigenic carrier and an antigenic compound, wherein the carrier is associated with the antigenic compound, thereby eliciting or enhancing an immune response to said compound.

51. The method of claim 50, wherein said association of the antigenic compound and the carrier is by charge-charge interaction.

52. The method of claim 50, wherein said association of the antigenic compound and the carrier is a covalent bond.

53. The method of claim 52, wherein said covalent bond is a peptide bond.

54. The method of claim 50, wherein said carrier is a polymer.

55. The method of claim 54, wherein said polymer is a polyamino acid polymer.

56. The method of claim 55, wherein said polyamino acid polymer is a polyanionic amino acid polymer.

57. The method of claim 56, wherein said polyanionic amino acid polymer is a polyglutamate polymer.

58. The method of claim 54, wherein said polymer is selected from the group consisting of polyanionic amino acids, polyglycolide, polylactide, poly(p-dioxanone), polycaprolactone, polyhydroxyalkanoates, poly(propylene fumarate), poly(ortho esters), polyanhydrides, polyphosphazenes, poly(alkylcyanoacrylates), poloxamers, polyglutamate, polyethylene glycol.

59. The method of claim 54, wherein said polymer is biodegradable.

60. The method of claim 54, wherein said polymer has a molecular weight of between ten and 5,000 KD.

61. The method of claim 60, wherein said polymer has molecular weight of between 100 and 1,000 KD.

62. The method of claims 61, wherein said polymer is about 600 KD.

63. The method of any one of claims 50 or 54, further comprising administering a physiologically acceptable excipient or diluent.

64. The method of any one of claims 50 or 54, further comprising administering an adjuvant.

65. The method of any one of claims 50 or 54, wherein said antigenic compound is derived from an agent selected from the group consisting of a virus, a fungus, a bacteria, a diseased tissue, and a hapten.

66. The method of claim 65, wherein said diseased tissue is a tumor.

67. The method of claim 65, wherein said agent is a virus.

68. The method of claim 65, wherein said agent is a fungus.

69. The method of claim 65, wherein said agent is a bacterium.

70. The method of claim 65, wherein said agent is a hapten.

71. The method of claim 50, wherein said antigenic compound is selected from the group consisting of peptides, proteins, polysaccharides, and haptens.

72. A mammal inoculated with the composition of any one of claims 1, 5, 19, 39, or 43.

73. A kit comprising a substantially non-antigenic carrier and instructions for associating said carrier with an antigenic compound.

74. The kit of claim 73, wherein said carrier is a polymer.

75. The kit of claim 74, wherein said polymer is a polyanionic amino acid polymer.

76. The kit of claim 75, wherein said polyanionic amino acid polymer is a homopolymer.

77. The kit of claim 76, wherein said homopolymer is a polyglutamate homopolymer.

78. The kit of claim 77, wherein said homopolymer has a molecular weight of between ten and 5,000 KD.

79. The kit of claim 78, wherein said polymer has molecular weight of between 100 and 1,000 KD.

80. The kit of claim 79, wherein said polymer is about 600 KD.

81. A method for inducing or enhancing an immune response in a mammal, comprising administering to said mammal an effective amount of a composition of any one of claims 1, 5, 19, 39, or 43.

82. The method of claim 81, wherein said immune response is a cellular immune response.

83. The method of claim 81, wherein said immune response is a humoral immune response.

84. A method for treating or ameliorating a viral infection in a mammal, comprising administering to said mammal an effective amount of a composition of any one of claims 1., 5, 19, 39, or 43.

85. A method for treating or ameliorating a pathology in a mammal, comprising administering to said mammal an effective amount of a composition of any one of claims 1, 5, 19, 39, or 43.
